(19) United States
(12) Patent Application Publication
(10) Pub. No.: US 2018/0071024 A1
(43) Pub. Date: Mar. 15, 2018

(54) ENHANCED EFFICACY AND TARGETED TOPICAL DELIVERY OF DRUGS TO SKIN WITH NANOPARTICLES

(71) Applicant: Sienna Biopharmaceuticals, Inc., Vestlake Village, CA (US)

(72) Inventor: Todd James Harris, Carlsbad, CA (US)

(21) Appl. No.: 15/559,397

(22) PCT Filed: Mar. 18, 2016
(86) PCT No.: PCT/US16/23174

§ 371 Date: Sep. 18, 2017

Related U.S. Application Data
(60) Provisional application No. 62/135,908, filed on Mar. 20, 2015.

(51) Int. Cl. A61B 18/20; A61K 41/00; A61K 8/02; A61N 5/06; A61K 8/19

(52) U.S. Cl. CPC A61B 18/203; A61K 41/0052; A61K 8/02; A61B 2018/0047; A61K 8/19; A61N 5/0616; A61N 5/0625; A61N 5/062

(57) ABSTRACT

Embodiments of the invention relate to enhanced efficacy of agents (such as drugs) for treatment of skin tissue with photoactive particles, such as plasmonic nanoparticles, and light. Enhanced efficacy may result from, for example, enhanced delivery, activity, or release of the drug that is caused by the localized heat generated via the nanoparticles. Treatments are useful for cosmetic, diagnostic and therapeutic applications.

### PRIORITY CLAIM AND INCORPORATION BY REFERENCE

This application claims the benefit of priority from U.S. Application 62/135,908 filed on Mar. 20, 2015, which is hereby incorporated by reference in its entirety herein.

### BACKGROUND

### Field of the Invention

The field of the invention comprises improved efficacy and delivery of agents, such as drugs, with photoactive compounds (such as nanoparticles) for use in cosmetic, diagnostic and/or therapeutic procedures to targeted structures on and/or within the skin or other target tissue.

### Description of the Related Art

Compositions, such as drugs for treating a skin tissue and/or tissue under a skin surface have demonstrated effectiveness through topical application. Light (e.g., laser, intense pulsed light (IPL), etc.) treatments of the skin have been touted for therapeutic and cosmetic utility.

Nanoparticles are disclosed in the art for dermatological purposes including but not limited to those disclosed in patents and publications to Harris et. al.

### SUMMARY

In various embodiments, the invention relates to improved efficacy and delivery of agents (e.g., drugs or other compounds), with nanoparticles and/or photoactive compounds for use in cosmetic, diagnostic and therapeutic procedures to targeted structures (e.g., pores, pilosebaceous units, sebaceous glands, hair follicles, scars, moles, freckles, acne, etc.) on or within the skin. Gold or silver nanoparticles are provided in combination with anti-acne agents in some embodiments. Several embodiments to process hair are also provided (such as coloring, straightening or curling), wherein the composition(s) are applied to the hair and/or scalp.

According to several embodiments of the invention, the nanoparticles are used to localize heating in a target region (such as a pilosebaceous unit). The localized heat, in turn, activates an agent, such as a drug or other compound. The agent, in one embodiment, can be “activated” through its release such that the heat causes a coating or encapsulation layer to partially or fully dissolve. The agent, in another embodiment, can also be “activated” because the agent itself is a heat-activated compound. In several embodiments, encapsulated release is linked to the particle (e.g. on a shell or coating layer). For example, an encapsulated agent may be chemically bonded or otherwise linked to a photoactive particle. In several embodiments, encapsulated release is separate from the particle (e.g. in a separate particle). For example, in one embodiment, an encapsulated agent may be freely floating, and separate and unassembled from a photactive particle.

In several embodiments, the invention comprises both professional and consumer use. With the latter, an at-home system is provided using a hand-held light source or wearable (such as a mask) to achieve localized heat generation of photoactive particles (e.g., nanoparticles).

Although several aesthetic and dermatology examples are provided herein, the combination of nanoparticles (or other photoactive materials) with agents can be used for other indications, including oncology.

In various embodiments, the invention comprises photoactive particles (e.g., nanoparticles) for use in cosmetic, diagnostic and therapeutic procedures. The photoactive particles (e.g., nanoparticles) are used in combination with agents to achieve a synergistic effect that enhances the permeability, absorption, activity, delivery, localization or release of the agent. Agents include, but are not limited to, drugs, solutions, compounds, small molecule formulations, hormones, vaccines, cosmetic toxins, cosmetics, cosmeceuticals, biologics, and biological agents. In one embodiment, the methods disclosed herein are useful for increasing the effectiveness and/or delivery of agents to a target tissue. In several embodiments, this increase is particularly advantageous because the enhanced effectiveness of the agent (based on the localized heat of the nanoparticles) reduces the concentration (and thus undesired effects) of the agent by more than 10-50% as compared to using the agent alone. In various embodiments, the amount of agent needed to achieve the same effect is reduced by at least 20, 30, 40, 50, or 60 percent or more when used in conjunction with the nanoparticle embodiments as described herein. In one embodiment, the amount of agent is either reduced or remains the same; however the time it takes for the agent to perform its function (when used with nanoparticles) is reduced by 10-90% as compared to using the agent alone. For example, if an agent would normally take 15 minutes to achieve an exfoliating, skin whitening, lightening or brightening or other effect, the agent combined with the nanoparticles would take 1-5 minutes. The reduction in time reduces side effects and/or shortens the duration of the treatment, which in turn enhances patient comfort.

In some embodiments, a sufficient amount of therapeutic material is activated by photoactive particles (including but not limited to plasmonic nanoparticles). In some embodiments, a therapeutically effective amount of material is released from encapsulation via heating by photoactive particles (including but not limited to plasmonic nanoparticles). In some embodiments, the particles are unassembled. In some embodiments, the agent is a pharmaceutical. In some embodiments, the agent is a cosmetic. In some embodiments, a therapeutically effective amount is an amount of an agent that produces a desired effect. In some embodiments, a therapeutically effective amount is an amount of a material that produces tissue repair, healing or cosmetic effect in a patient or subject.

In several embodiments, the invention relates to using laser or light energy combined with photoactive particles (e.g., nanoparticles) with an agent (e.g., drug treatment) to treat, modify, smooth, and/or resurface the skin (including tissue under the skin surface) of humans. In various embodiments, a plasmonic nanoparticle composition is used to selectively target structures in the skin tissue to heat and/or selectively damage tissue with exposure to energy. In various embodiments, heating of tissue with the nanoparticles increases the temperature of a targeted tissue in the vicinity and/or in contact with the nanoparticles by 1, 2, 3, 4, 5, 7, 10, 15, 20, 25, 30, 35, 40, 45, 50, 55, 60, 65, 70, 75, 80 degrees Celsius, or more, and any temperature ranges therein (e.g., by 2-10 degrees).

In some embodiments, non-heat transfer of energy (e.g., Förster resonance energy transfer (FRET), resonance energy transfer (RET), electronic energy transfer (EET)) can be used as an alternative means to enhance the activity of photoreactive compounds. In some embodiments, energy is donated from one photoreactive compound and transferred to a receiving, or accepting compound. This type of energy transfer can be sensitive to the distance between the compounds, particles, etc.

In some embodiments, the tissue is cooled before or after heat is generated to facilitate the regulation of temperature. In some embodiments, the tissue is cooled by 1, 2, 3, 4, 5, 7, 10, 15, 20, 25, 30, 35, 40, 45, 50, 55, 60, 65, 70, 75, 80 degrees Celsius, or more, and any temperature ranges therein (e.g., by 2-10 degrees). In some embodiments, a cooling, cryonic, chilling, and/or freezing device is used before, during, and/or after a treatment. In various embodiments, a plasmonic nanoparticle composition is used to selectively target structures in the skin tissue to increase permeability of the tissue at the localized target structure. In various embodiments, a plasmonic nanoparticle composition is used to selectively target structures in the skin tissue to increase kinetics of biochemical processes in or near the tissue at the localized target structure. As an example, in one embodiment, localized heating is provided to dissolve an encapsulation material and cooling is subsequently provided for either patient comfort reasons and/or because the agent functions better at the lower temperature.

In several embodiments, the invention relates to using laser or light energy combined with photoactive particles (e.g., nanoparticles) in conjunction with (e.g., before, during/concomitant with, or after) an agent (e.g., drug treatment) to treat the skin (including tissue under the skin surface) by improving the efficacy and/or facilitating the delivery of the agent (e.g., drug) with plasmonic nanoparticles and/or photoactive compounds at the target tissues. In various embodiments, the invention is able to selectively target specific tissue, increase tissue permeability, improve kinetics, increase reaction rates, increase chemical interactions for treatment, and/or modify, smooth, and/or resurface the skin (including tissue under the skin surface) of humans. In some embodiments, permeability of the tissue and/or the drug kinetics are increased by about 10-1000% (e.g., 25-100%, 50-200%, 100-400%, 250-350%, 200-500%, 50-300%, 500-750%, 100-900%, and overlapping ranges therein). In some embodiments, the permeability and/or kinetics are increased by a factor of at least 2× to 10× (e.g., 2×-4×, 3×-5×, 2×-8×, and overlapping ranges therein). In some embodiments, the improvements are non-linear. In some embodiments, the improvements are exponential. Several embodiments of the present invention also relate to methods for focusing electromagnetic energy with particles and/or photoactive compounds to selectively heat target regions of skin with a continuous or pulsed light treatment. In various embodiments, the irradiation is provided continuously. In various embodiments, the irradiation is pulsed. In various embodiments, the pulses can be provided in 0.1, 0.5, 1, 2, 3, 4, 5, 10, 15, 20, 30, 40, 50, 75, 100, 200, 500, and 1000 ms, or any range therein. In some embodiments, the pulsed irradiation is provided in a range of 5-30 ms, 1-50 ms, 5-10 ms, and/or 20-100 ms, and any ranges therein.

In several embodiments, the invention relates to using laser or light energy combined with plasmonic nanoparticles and/or other photoactive compounds to release an agent (e.g., drug) that is encapsulated in a heat or temperature responsive material. In some embodiments, an encapsulated agent may be bonded directly to a photoactive particle. In some embodiments, an encapsulated agent is separate, and unassembled with a photoactive particle. Heating of the plasmonic nanoparticles controls the rate and extent of agent (e.g., drug) released by the encapsulation in some embodiments. In various embodiments, heating of the agent (e.g., drug) encapsulation material with the nanoparticles increases the temperature of encapsulation material in contact with the nanoparticles by 1, 2, 3, 4, 5, 7, 10, 15, 20, 25, 30, 35, 40, 45, 50, 55, 60, 65, 70, 75, 80 degrees Celsius, or more, and any temperature ranges therein (e.g., by 2-10 degrees). In various embodiments, the encapsulation material comprises a release temperature such that heating the encapsulation material above the release temperature results in opening the encapsulation (by, for example, dissolving all or part of the encapsulation, creating one or more pores in the encapsulation, etc.). In various embodiments, the release temperature can be 1, 2, 3, 4, 5, 7, 8, 9, 10, 11, 12, 13, 14, 15, 20, or 25 degrees Celsius (and any temperature values therein) above a subject's body temperature (e.g., by 2-10 degrees). In some embodiments, the release temperature subjects the drug encapsulation material to a phase change (e.g., solid to liquid, etc.). In various embodiments, the drug encapsulation material comprises a polymer and/or a matrix material.

Also, provided herein, in several embodiments, are compositions and methods useful in the targeted heating and/or thermomodulation of target cell populations and target tissues, for the purposes of medical, cosmetic, and/or other treatments and prevention of chronic and acute diseases and disorders and enhanced delivery systems and methods (e.g., for improving the delivery and effectiveness of drugs and/or compounds to a target tissue.

In various embodiments, a method of increasing kinetics of a drug reaction with a composition at a target tissue under a skin surface, includes applying a composition to a skin surface, distributing the composition from the skin surface to a target tissue under the skin surface, wherein said composition comprises a plurality of photoactive particles (e.g., plasmonic nanoparticles), wherein the photoactive particles comprise a coating. In one embodiment, the photoactive particle has a metal portion and the coating is less conductive than the metal portion. The coating facilitates selective removal from the skin surface, selectively removing the composition from the skin surface, while leaving the composition localized within the target tissue, applying a drug to a skin surface, and irradiating the composition with a visible spectrum light source and/or infrared light source thereby inducing a plurality of surface plasmons, wherein inducing the plurality of surface plasmons generates localized heat in the target tissue, thereby increasing a temperature of the target tissue, thereby increasing kinetics of a drug reaction at the target tissue. Several embodiments to process hair are also provided (such as coloring, straightening or curling), wherein the composition(s) are applied to the hair and/or scalp.

The visible light source includes, for example, violet 380 nm-450 nm wavelength, blue 450 nm-495 nm wavelength, green 495 nm-570 nm wavelength, yellow 570 nm-590 nm wavelength, orange 590 nm-620 nm wavelength, red 620 nm-750 nm wavelength, 380 nm-750 nm, 350 nm-700 nm, 400 nm-450 nm, 410 nm-440 nm, 440 nm, 620 nm-700 nm, 630 nm-660 nm, 640 nm, and overlapping ranges and values therein. The infrared light source includes, for example, 700 nm-1 mm wavelengths, 700 nm-1200 nm, 750 nm-1200 nm, 700 nm, 755 nm, 800 nm, 810 nm, 1064 nm, 1200 nm, etc. and overlapping ranges and values therein.

In various embodiments, the invention comprises methods of using photoactive particles such as plasmonic nanoparticles for one or more of the following: (i) increasing kinetics of a drug reaction at a target tissue under a region (e.g., skin surface), (ii) increasing the permeability of an agent or tissue, such as skin, and/or (iii) wound healing. These methods, according to several embodiments, include: applying a composition to a region (e.g., skin surface), distributing the composition from the skin surface to a target tissue under the skin surface (e.g., under, near, around, proximate), selectively removing the composition from the skin surface while leaving the composition localized within the target tissue, applying an agent (e.g., drug) to the target tissue, and irradiating the composition with a light source. In one embodiment, the photoactive particles comprise a conductive metal portion and a coating. In one embodiment, the coating is less conductive than the metal portion. In some embodiments, the conductivity of the coating or other layer is less than 1%, less than 10% or less than 75% of the conductivity of the metal portion. In one embodiment, the coating facilitates selective removal from the skin surface. In one embodiment, irradiating the composition with a light source induces a plurality of surface plasmons wherein inducing the plurality of surface plasmons generates localized heat in the target tissue, thereby increasing a temperature of the target tissue, thereby (i) increasing kinetics of a drug reaction at the target tissue, (ii) increasing permeability of a drug reaction at the target tissue, and/or (iii) enhancing wound healing.

Although the skin surface is used herein as an example, several embodiments also contemplate apply this method to a region that is not a skin surface, but rather a surface or other area of a tissue.

In several embodiments, the conductive metal portion comprises at least one of gold and silver, the coating is hydrophilic, and the light source comprises a wavelength selected 400 to 1200 nm (e.g., 440 nm, 640 nm, 750 nm, 800 nm, 810 nm, and 1064 nm). The conductive metal portion can be gold and the drug can be an anti-acne drug (e.g., glycolic acid, sulfur, salicylic acid, benzoyl peroxide or other anti-acne drug), and optionally the coating can be polyethylene glycol (PEG) or silica. Instead of gold or silver, platinum can be used. In one embodiment, the plasmonic nanoparticles have a concentration from 10⁹-10¹³ (e.g., 10⁹, 10¹⁰, 10¹¹, 10¹², or 10¹³) particles per ml of the composition (which can be a solution, gel, ointment, salve, lotion, etc.). In one embodiment, the plasmonic nanoparticles (which can be unassembled nanoparticles) have at least one dimension in the range of 50-200 nm (e.g., 100 nm to 200 nm). In one embodiment, the temperature of the target tissue effects kinetic reactions is based on the Arrhenius equation. The temperature of the target tissue can be increased by 1-30 (e.g., 1-5, 5-10, 5-20, 10-30, etc.) degrees Celsius. In one embodiment, the light source irradiates the composition with a pulse of in a range of 1 ms to 30 ms (e.g., 2-8, 5-25, 18-26 ms, etc.). Optionally, distributing the composition comprises the use of low frequency ultrasound. In one embodiment, the plasmonic nanoparticles comprise an optical density of 10 O.D. to 500 O.D. (e.g., 50, 100, 125, 150, 200, 250, 300 O.D.). In one embodiment, the plasmonic nanoparticles comprise a solid, conducting silver core and a silica coating. In one embodiment, the conductive metal portion is a nanoplate, rod or shell (e.g., silver nanoplate, gold rod, gold nanoshell, etc.), and the coating aids in removing the nanoparticles from the skin (or other) surface.

In one embodiment, the composition comprises any one or more of a biologic, a biological agent, a humectant, a surfactant, a thickener, a dye, an antiseptic, an anti-inflammatory agent, an anti-oxidant, a vitamin, a fragrance, an oil, an adhesive, and a topical anesthetic. In one embodiment, the composition comprises any one or more of growth factors, collagen byproducts, collagen precursors, hyaluronic acid, glucosamine, allantoin, vitamins, oxidants, antioxidants, amino acids, retinoids, retinoid-like compounds, and minerals. In one embodiment, the drug comprises any one or more of steroidal anti-inflammatory drugs, non-steroidal anti-inflammatory drugs, antioxidants, antibiotics, antiviral drugs, antiyeast drugs and antifungal drugs. In one embodiment, the method also includes pre-treating the skin surface prior to irradiating the composition, wherein pre-treating the skin surface comprises hair removal.

In one embodiment, the agent comprises any one or more of a biologic, a biological agent, a humectant, a surfactant, a thickener, a dye, an antiseptic, an anti-inflammatory agent, an anti-oxidant, a vitamin, a fragrance, an oil, an adhesive, and a topical anesthetic. In one embodiment, the drug comprises any one or more of growth factors, healing factors, collagen byproducts, collagen precursors, hyaluronic acid, vitamins, antioxidants, amino acids, retinoids, retinoid-like compounds (e.g., retinol), and minerals. In some embodiments, hyaluronic acid is provided in an amount sufficient to effectively assist in tissue repair. In some embodiments, hyaluronic acid is provided in an amount sufficient to effectively treat a tumor or lesion. In some embodiments, hyaluronic acid is provided in an amount sufficient to regenerate tissue, e.g., skin tissue, organ tissue, etc. In one embodiment, the drug comprises any one or more of steroidal anti-inflammatory drugs, non-steroidal anti-inflammatory drugs, antioxidants, antibiotics, antiviral drugs, antiyeast drugs and antifungal drugs. In one embodiment, the drug comprises any one or more of glycolic acid, sulfur, salicylic acid, and benzoyl peroxide. In some embodiments, a composition comprises a peroxide (e.g., benzoyl peroxide, hydrogen peroxide, etc.). In some embodiments, a composition comprises a peroxide in a concentration of 1-20% (e.g., 1, 2, 3, 4, 5, 10, 12, 15, 18, 20% and any range or amount therein) per ml of the composition, or % m/m, % m/v, or % v/v of the composition. In some embodiments, a peroxide is provided in a concentration effective to kill bacteria. In some embodiments, a peroxide is provided in a concentration effective to reduce acne.

In several embodiments, the agents disclosed herein are provided in a therapeutically effective range. In some embodiments, the range is about 0.05-25% (e.g., 0.05-5, 1-3, 2-5, 4-9%, 10-25% and overlapping ranges therein) per ml of the composition, or % m/m, % m/v, or % v/v of the composition. Lower or higher amounts and concentrations are provided in other embodiments to achieve a therapeutic effect. The amounts of the agents and particles described herein are selected to achieve a therapeutic effect. In some embodiments, the amount of agent used in combination with the photoactive (e.g., nanoparticle) technology described herein is 10-75% less than used alone (to achieve a similar effect). As a non-limiting example, if the commercially-available concentration of a particular topical agent is 3%, that agent may be used in combination with the photoactive particles described herein at a concentration of 0.05-2% to achieve the same effect. In several embodiments, the combination of the agent and photoactive particles (e.g., nanoparticles) described herein are more effective than using either alone and show, in several embodiments, synergistic effects.

In one embodiment, the temperature of the target tissue effects kinetic reactions based on the Arrhenius equation. In one embodiment, the temperature of the target tissue is increased by 1-30 degrees Celsius (e.g., 1, 2, 3, 4, 5, 7, 10, 15, 20, 25, 30 degrees Celsius, and any ranges or values therein). In one embodiment, the temperature of the target tissue is increased by 5-15 degrees Celsius (e.g., 5, 6, 7, 8, 9, 10, 11, 12, 13, 14, 15 degrees Celsius, and any ranges values therein). In one embodiment, the infrared light source irradiates the composition with a pulse of in a range of 1 ms to 30 ms. In one embodiment, the distributing the composition comprises activation of a mechanical vibration device is a low frequency ultrasound device, bubble formation, or liquid microstreaming.

In one embodiment, the nanoparticle composition (e.g., a drug formulation) is used as a topical composition in the treatment of an infection. In various embodiments, any of the materials and/or compositions from any embodiment herein is used. In one embodiment, about 1 ml of the composition will be applied to the skin proximate an infection once a day or once a week (or other treatment frequency) until the infection symptoms subside. In one embodiment a therapeutically effective amount of a material will be applied to an infection. Plasmonic nanoparticles are applied to the infection. Energy, e.g., infrared light, is applied to activate a plasmon in the plasmonic nanoparticles, thereby increasing the effectiveness of the material applied to the infection, thereby treating the infection. In one embodiment, a material is encapsulated with a nanoparticle and applied to an infection site. Energy, e.g., infrared light, is applied to activate a plasmon in the plasmonic nanoparticles, thereby activating the material applied to the infection, thereby treating the infection. In some embodiments, the nanoparticles are coated with a hydrophilic coating. In some embodiments, the nanoparticles and material are in a composition with a cosmetically and/or pharmacologically acceptable carrier. In one embodiment, the nanoparticle comprises gold as an inner component, outer component, or both. In one embodiment, the nanoparticle comprises silver as an inner component, outer component, or both. In one embodiment, the nanoparticles are solid metal particles with an outer coating comprising silica or PEG. In one embodiment, the nanoparticles are non-metal particles with an outer component comprising silver or gold. In several embodiments, the nanoparticles have a concentration of 10⁹ to 10¹⁴ per ml of the composition (e.g., 10⁹, 10¹⁰, 10¹¹, 10¹², 10¹³, 10¹⁴ and overlapping ranges therein).

In one embodiment, plasmonic nanoparticles and a therapeutic material are dissolved in a solution and applied to an infection site to treat the infection site with the application of light and/or heat energy. In some embodiments, the light has a visible spectrum wavelength (e.g., 380 nm-750 nm, 350 nm-700 nm, 400 nm-450 nm, 410 nm-440 nm, 440 nm, 620 nm-700 nm, 630 nm-660 nm, 640 nm, and overlapping ranges therein). In some embodiments, the light has a violet wavelength (e.g., 380 nm-450 nm, 390 nm-440 nm, 420 nm, 430 nm, 440 nm, and overlapping ranges therein). In some embodiments, the light has a red wavelength (e.g., 620 nm-750 nm, 630 nm-700 nm, 640 nm-660 nm, 640 nm, 650 nm, and overlapping ranges therein). In some embodiments, the light has an infrared wavelength (e.g., 700-1200 nm, 700 nm, 755 nm, 800 nm, 810 nm, 1064 nm, 1200 nm, etc. and overlapping ranges therein). In some embodiments, the light source is a device (e.g., lamp, bulb, laser, LED, etc.). In some embodiments, the light source is naturally occurring (e.g., the sun). In some embodiments, the composition comprises organic material. In some embodiments, the composition comprises inorganic material. In some embodiments, the composition comprises a solid. In some embodiments, the composition comprises a liquid. In some embodiments, the composition comprises a gas. In some embodiments, the composition comprises bubbles. In some embodiments, the composition is subjected to a phase change. In some embodiments, the composition is stirred. In some embodiments, the composition is centrifuged. In some embodiments, the composition is filtered. In some embodiments, the composition is thickened. In some embodiments, the composition is thinned. In some embodiments, the composition has increased viscosity. In some embodiments, the composition has decreased viscosity. In some embodiments, the composition is refined. In some embodiments, the composition is stabilized. In one embodiment, mechanical vibration is used to assist in the targeted delivery of the composition to an infection site for treatment. In one embodiment, acoustic vibration is used to assist in the targeted delivery of the composition to an infection site for treatment. In one embodiment, ultrasound is used to assist in the targeted delivery of the composition to an infection site for treatment. In one embodiment, suction is used to assist in the targeted delivery of the composition to an infection site for treatment. In one embodiment, air pressure is used to assist in the targeted delivery of the composition to an infection site for treatment.

In various embodiments, a method of using nanoparticles for cosmetically treating one or more dysfunctional pilosebaceous units at a target tissue under a skin surface includes applying the composition to the skin surface, distributing the composition from the skin surface to the target tissue under the skin surface, selectively removing the composition from the skin surface, while leaving the composition distributed to the target tissue at a concentration sufficient for cosmetically treating the one of more dysfunctional pilosebaceous units, applying the one or more drugs to the target tissue at a concentration sufficient for cosmetically treating the one of more dysfunctional pilosebaceous units, and irradiating the composition at the target tissue with a light source. In one embodiment, the composition comprises a plurality of plasmonic nanoparticles. In one embodiment, the plasmonic nanoparticles comprise a metal portion and a coating, wherein the coating is less conductive than the metal portion, wherein said coating facilitates selective removal of the composition from the skin surface. In one embodiment, irradiating the composition at the target tissue with a light source providing a light energy induces a plurality of surface plasmons in said plasmonic nanoparticles, wherein inducing the plurality of surface plasmons activates the one or more drugs at the target tissue and promotes a healing of the one or more dysfunctional pilosebaceous units, thereby cosmetically treating the one or more dysfunctional pilosebaceous units.

In various embodiments, a method of increasing kinetics of a drug reaction and/or increasing skin permeability with a transdermal patch includes applying a transdermal patch comprising a composition to a region (e.g., skin surface), wherein said composition comprises a drug and a plurality of photoactive particles such as plasmonic nanoparticles. In one embodiment, the plasmonic nanoparticles comprise a metal. In one embodiment, the transdermal patch comprises a portion configured for transmission of light through the transdermal patch. The method includes irradiating the composition with a light source thereby inducing a plurality of surface plasmons in said plasmonic nanoparticles, wherein inducing the plurality of surface plasmons generates localized heat in a target tissue, thereby increasing a temperature of the target tissue, thereby increasing kinetics of a drug reaction and/or permeability at the target tissue. Alternatively, the method includes irradiating the composition with a light source thereby inducing the photoactive particles to produce localized heat in a target tissue, thereby increasing a temperature of the target tissue, thereby increasing kinetics of a drug reaction at the target tissue. In other embodiments, the transdermal patch is used for healing a wound.

In various embodiments, a transdermal patch includes a transmission portion configured for transmission of light through the transdermal patch, an adhesive configured for application of the transdermal patch to a skin surface, a composition comprising a drug and a plurality of plasmonic nanoparticles, wherein the transmission portion is configured for transmission of sufficient light to induce a plurality of surface plasmons in said plasmonic nanoparticles to generate localized heat in a target tissue proximate the skin surface, thereby increasing a temperature of the target tissue, thereby increasing kinetics of a drug reaction at the target tissue. In one embodiment, the transmission portion is configured to transmit between 80% and 100% (e.g., 80, 85, 90, 95, 98%) of light through the transdermal patch. In one embodiment, the transmission portion comprises an image of a treatment region on the skin surface. In one embodiment, the transmission portion comprises an outline of a treatment region. The patch can be pre-populated with the particles, the agent(s) or both. Alternatively, the patch can be placed on skin after application of particles and/or agent(s). In yet other embodiments, the patch is porous and allows placement on the skin before adding the particles and/or agents on top of the patch.

In one embodiment, the composition comprises any one or more of growth factors, collagen byproducts, collagen precursors, hyaluronic acid, glucosamine, allantoin, vitamins, oxidants, antioxidants, amino acids, retinoids, retinoid-like compounds, and minerals. In one embodiment, the composition comprises any one or more of steroidal anti-inflammatory drugs, non-steroidal anti-inflammatory drugs, antioxidants, antibiotics, antiviral drugs, antiyeast drugs and antifungal drugs. In one embodiment, the drug comprises any one or more of glycolic acid, sulfur, salicylic acid, and benzoyl peroxide. In one embodiment, the temperature of the target tissue effects kinetic reactions based on the Arrhenius equation. In one embodiment, the temperature of the target tissue is increased by 1-30 degrees Celsius (e.g, 1, 5, 10, 15, 20, 25 degrees). In one embodiment, the temperature of the target tissue is increased by 5-15 degrees Celsius (e.g., 6, 8, 11, 13 degrees). In one embodiment, the light source irradiates the composition with a pulse of in a range of 1 ms to 30 ms (e.g., 2, 4, 8, 12, 14, 17, 22, 26, 29 ms). In one embodiment, the distributing the composition comprises activation of a mechanical vibration device with low frequency ultrasound on the transdermal patch. In one embodiment, the distributing the composition comprises activation of a mechanical vibration, further comprising pre-treating the skin surface prior to applying of the transdermal patch on the skin surface, wherein pre-treating the skin surface comprises hair removal. In one embodiment, the distributing the composition comprises activation of a mechanical vibration, wherein the plasmonic nanoparticles comprise an optical density of 10 O.D. to 500 O.D. at a light range from 700 nm to 1200 nm. In one embodiment, the distributing the composition comprises activation of a mechanical vibration, wherein the plasmonic nanoparticles comprise a solid, conducting silver core and a silica coating. In one embodiment, the metal portion is conductive, the metal portion is a silver nanoplate, and the coating is less conductive than the metal portion. In one embodiment, the drug is provided in a therapeutically or cosmetically effective amount. In one embodiment, the drug comprises salicylic acid. In one embodiment, the drug comprises benzoyl peroxide. In one embodiment, the drug comprises glycolic acid. In one embodiment, the drug comprises sulfur. In one embodiment, the drug comprises 5-ALA.

In one embodiment, the composition is distributed from the skin surface to one or more dysfunctional pilosebaceous units at a target tissue by massaging the composition by hand or with a mechanical vibration device. In one embodiment, the mechanical vibration device vibrates at a range of about 50 Hz to about 100 Hz. In one embodiment, the mechanical vibration device vibrates at about 80 Hz. In one embodiment, the light source is a diode and the light energy is in the violet, red, or infrared range. In various embodiments, the light wavelength is 440 nm, 640 nm, 750 nm, 800 nm, 810 nm, or 1064 nm. In one embodiment, the light energy is provided by an Intense Pulsed light (IPL) device operating at 1-20 J/cm² and 1-5 ms pulse width. In one embodiment, the skin surface is on the face, neck, head, body, chest, back or a combination thereof. In one embodiment, cosmetically treating the one or more dysfunctional pilosebaceous occurs over the course of about 1 week to about 10 weeks. In various embodiments, the plasmonic nanoparticles comprise one or more of nanoplates, nanorods, hollow nanoshells, silicon nanoshells, nanorice, nanowires, nanopyramids and nanoprisms. In one embodiment, the nanoparticle is not a nanoshell. In several embodiments, the nanoparticle is a nanoplate. In several embodiments, the nanoplates are manufactured according to the manufacturing methods of U.S. Pat. No. 9,212,294, hereby incorporated by reference. In one embodiment, the plasmonic nanoparticles have a size at least in one dimension in a range of about 1 nm to about 1000 nm (e.g., 20-100, 100-200, 200-300, 300-500 nm, and overlapping ranges therein). In some embodiments, the photoactive particles are sized to fit (i) within a pilosebaceous unit at concentrations between 10⁹-10¹⁶ or (ii) within a target tissue sized up to 900 microns at concentrations between 10⁹-10¹⁶. Photoactive particles can also be much larger than nanoparticles (e.g., by several fold).

In one embodiment, the plasmonic nanoparticles have a size at least in one dimension in a range of about 10 nm to about 300 nm. In one embodiment, the one or more drugs comprises one or more of glycolic acid, sulfur, salicylic acid and benzoyl peroxide. In one embodiment, the light energy causes an increase in temperature by heating of the one or more pilosebaceous units, wherein an increase in temperature by heating causes an increase in the permeability of the target tissue for the one or more drugs, and wherein the increase in temperature by heating causes an increase in a reaction rate of the one or more drugs at the target tissue. In one embodiment, the increase in the reaction rate of the one or more drugs at the target tissue caused by the increase in temperature by heating is determined based on the Arrhenius equation. In one embodiment, the increase in temperature of the target tissue is by about 1 degree Celsius to about 30 degrees Celsius. In one embodiment, the increase in temperature of the target tissue is by about 5 degrees Celsius to about 15 degrees Celsius. In one embodiment, the concentration sufficient for cosmetically treating the one of more dysfunctional pilosebaceous of the composition is about 10⁹ to about 10¹⁶ plasmonic nanoparticles per ml of the composition (e.g., 10⁹, 10¹⁰, 10¹¹, 10¹², and 10¹³ particles per ml of the composition).

In various embodiments, a composition comprising a plurality of photoactive particles and an anti-acne agent is provided. In one embodiment, the composition includes a plurality of photoactive particles (such as plasmonic nanoparticles) comprising a conductive metal portion (e.g., gold, silver, platinum) and a coating (e.g., PEG, silica), wherein the coating is less conductive than the metal portion, wherein the coating is configured to facilitate selective removal from a tissue surface, wherein the conductive metal portion comprises at least one of gold and silver, wherein the coating is hydrophilic, wherein the plasmonic nanoparticles have a peak absorption wavelength selected from the group consisting of: 440 nm, 640 nm, 750 nm, 800 nm, 810 nm, and 1064 nm, wherein the plasmonic nanoparticles are configured to induce a plurality of surface plasmons at the peak absorption wavelength to generate localized heat, and an anti-acne agent. In one embodiment, the particles have a concentration selected from the group consisting of: 10⁹, 10¹⁰, 10¹¹, 10¹², and 10¹³ particles per ml of the composition and at least one dimension in the range of 100 nm to 200 nm. In one embodiment, the anti-acne agent is selected from the group consisting of hyaluronic acid, glycolic acid, salicylic acid, sulfur, and benzoyl peroxide.

Several embodiments to process hair are also provided (such as coloring, straightening or curling), wherein the composition(s) are applied to the hair and/or scalp. Anti-seborrheic agents are provided in several embodiments to treat hair and skin (e.g., scalp, face, underarms). These anti-seborrheic agents include, but are not limited to, salicylic acid, corticosteroids, selenium sulfide, zinc pyrithione, and imidazole antifungals, and combinations thereof. When one or more anti-seborrheic agents are used with photoactive particles described herein, a more efficient treatment for seborrhea (e.g., seborrheic dermatitis of the scalp, face, or other parts of the body, dandruff, etc.). In various embodiments, a composition comprising a plurality of photoactive particles and a hair treatment agent is provided. In one embodiment, the composition includes a plurality of plasmonic nanoparticles comprising a conductive metal portion and a coating, wherein the coating is less conductive than the metal portion, wherein the coating is configured to facilitate selective removal from a tissue surface, wherein the conductive metal portion comprises at least one of gold and silver, wherein the coating is hydrophilic, wherein the plasmonic nanoparticles have a peak absorption wavelength selected from the group consisting of: 440 nm, 640 nm, 750 nm, 800 nm, 810 nm, and 1064 nm, wherein the plasmonic nanoparticles are configured to induce a plurality of surface plasmons at the peak absorption wavelength to generate localized heat, and a hair treatment agent. In one embodiment, the particles are unassembled and the coating comprises silica or polyethylene glycol (PEG). In one embodiment, the particles have a concentration selected from the group consisting of: 10⁹, 10¹⁰, 10¹¹, 10¹², and 10¹³ particles per ml of the composition, and wherein the particles have at least one dimension in the range of 100 nm to 200 nm. In one embodiment, the hair treatment agent is selected from the group consisting of minoxidil, calcium hydroxide, sodium hydroxide, potassium thiogycolate, pigment, dye, hydrogen peroxide, and keratin.

In various embodiments, a composition comprising a plurality of photoactive particles and a skin lightening agent is provided. In one embodiment, the composition includes a plurality of plasmonic nanoparticles comprising a conductive metal portion and a coating, wherein the coating is less conductive than the metal portion, wherein the coating is configured to facilitate selective removal from a tissue surface, wherein the conductive metal portion comprises at least one of gold and silver, wherein the coating is hydrophilic, wherein the plasmonic nanoparticles have a peak absorption wavelength selected from the group consisting of: 440 nm, 640 nm, 750 nm, 800 nm, 810 nm, and 1064 nm, wherein the plasmonic nanoparticles are configured to induce a plurality of surface plasmons at the peak absorption wavelength to generate localized heat; and a skin lightening agent. In one embodiment, the particles are unassembled and the coating comprises silica or polyethylene glycol (PEG). In one embodiment, the particles have a concentration selected from the group consisting of: 10⁹, 10¹⁰, 10¹¹, 10¹², and 10¹³ particles per ml of the composition, and wherein the particles have at least one dimension in the range of 100 nm to 200 nm. In one embodiment, the skin lightening agent is selected from the group consisting of anti-melanin agents, hydroquinone, retinoic acid, and steroids.

The composition and methods summarized above and set forth in further detail below may recite a composition comprising particles and agent(s). This will be understood to mean that the particles (such as plasmonic nanoparticles) and the agent(s) can be pre-combined in a single container or can be provided in two or more containers. Simultaneous or sequential application of the particles and the agent(s) are contemplated.

In various embodiments, a kit is provided. The kit can include the photoactive particles and one or more agents described herein. The particles and the agent(s) can be pre-combined in a single container or can be provided in two or more containers. Instructions for use that provide simultaneous or sequential application of the particles and the agent(s) can be provided. In one embodiment, the kit includes a composition for the treatment of acne, hair, or skin lightening and a light source.

In several embodiments, the invention comprises the use of the compositions summarized above and set forth in further detail below for the treatment of dermatological conditions, such as acne, hair removal, skin lightening, as well as other uses as described herein.

The methods summarized above and set forth in further detail below describe certain actions taken by a practitioner; however, it should be understood that they can also include the instruction of those actions by another party. Thus, actions such as “identifying a target region” can include “instructing the identification of a target region” and “delivering an energy” can include “instructing the delivery of an energy.”

### BRIEF DESCRIPTION OF THE DRAWINGS

FIG. 1 is illustrative of schematics depicting certain embodiments of the use of compositions, solutions, drugs, and/or formulations for targeting of one or more structures in the skin, such as illustrated for a hair treatment and an acne treatment.

FIG. 2 is illustrative of a temperature profile of certain embodiments of the compositions of plasmonic nanoparticles (SL-001, triangles) provided herein compared to certain embodiments of clinical dyes carbon lotion (circles), meladine spray (diamonds), and indocyanine green (squares).

FIG. 3 is a schematic side view of a composition being distributed from a skin surface to a target in the tissue with a delivery device according to an embodiment of the invention.

### DETAILED DESCRIPTION

In various embodiments, the invention relates to improved efficacy and delivery of compositions, such as drugs, with photoactive particles (e.g., nanoparticles) for use in cosmetic, diagnostic and/or therapeutic procedures to one or more targeted structures (e.g., pores, pilosebaceous units, sebaceous glands, hair follicles, scars, moles, freckles, vascular/blood vessels, acne, tumors, etc.) on and/or within the skin. In some embodiments, the cosmetic, diagnostic and/or therapeutic procedure is directed to treating the condition itself. In some embodiments, the cosmetic, diagnostic and/or therapeutic procedure is directed to treating the symptoms and/or appearance of a condition, and/or the condition itself. For example, in some embodiments cosmetic, diagnostic and/or therapeutic procedures are directed to acne, hair, scars, skin discoloration, freckles, blemishes, age spots, melisma, pigmentation conditions, ageing of skin, wrinkles, diabetes, obesity, body shaping, orthopedic, neurological, cardiovascular, vascular, peripheral vascular and other conditions, infections, and other conditions. In some embodiments, the use of the compositions described herein is purely cosmetic and, for example, need not be performed by a physician.

In various embodiments, the invention comprises photoactive particles (e.g., nanoparticles) for use in cosmetic, diagnostic and/or therapeutic procedures, including use with one or more agents (such as a drug, a solution, a compound, a biologic, a biological agents, a humectant, a surfactant, a thickener, a dye, an antiseptic, an anti-inflammatory agent (e.g., hydrocortisone), an anti-oxidant, a vitamin, a fragrance, an oil, an adhesive, and/or a topical anesthetic, or combinations thereof). In various embodiments, the invention increases the effectiveness and/or delivery of those agents to a target tissue. In several embodiments, the invention relates to using light energy (e.g., laser, lamp, LED, natural light) combined with nanoparticles and/or photoactive compounds with an agent (a drug treatment and/or composition) to treat, modify, smooth, and/or resurface the skin (including tissue under the skin surface) of a mammal. In some embodiments, the mammal is human. In some embodiments, the mammal can be pet (e.g., companion animal to human) or a commercially beneficial animal to humans (e.g., dog, cat, sheep, goat, pig, cattle, horse, etc.).

In various embodiments, a plasmonic nanoparticle composition is used to selectively target structures in the skin tissue to increase permeability of the tissue at the localized target structure. In various embodiments, a plasmonic nanoparticle composition is used to selectively target structures in the skin tissue to increase kinetics of biochemical processes at or near the tissue at the localized target structure. In various embodiments, a plasmonic nanoparticle composition is used to selectively target structures in the skin tissue to increase permeability of the tissue and increase kinetics of biochemical processes at or near the tissue at the localized target structure. Several embodiments to process hair are also provided (such as coloring, straightening or curling), wherein the composition(s) are applied to the hair and/or scalp.

In various embodiments, the invention relates to using laser or light energy combined with plasmonic nanoparticles and/or other photoactive compounds to release an agent (such as a drug or other compound) that is encapsulated in a heat or temperature responsive material. Heating of the plasmonic nanoparticles controls the rate and extent of drug released by the encapsulation. In various embodiments, heating of the drug encapsulation material with the nanoparticles increases the temperature of drug encapsulation material in contact with the nanoparticles by 1, 2, 3, 4, 5, 7, 10, 15, 20, 25, 30, 35, 40, 45, 50, 55, 60, 65, 70, 75, 80 degrees Celsius, or more, and any temperature ranges therein. In various embodiments, the drug encapsulation material comprises a release temperature such that heating the drug encapsulation material above the release temperature results in opening the encapsulation. In various embodiments, the release temperature can be 1, 2, 3, 4, 5, 7, 8, 9, 10, 11, 12, 13, 14, 15, 20, or 25 degrees Celsius (and any temperature values therein) above a subject's body temperature. In some embodiments, the release temperature subjects the drug encapsulation material to a phase change (e.g., solid to liquid, etc.). In some embodiments, the encapsulation material or thermally-responsive coating is dissolved by increasing its temperature by 5-20% higher (e.g., 5%, 7%, 9%, 10%, 12%, 15%, 18%, 29%) than body temperature. In some embodiments, the encapsulation material or thermally-responsive coating is dissolved by increasing its temperature to 40-60 degrees Celsius (e.g., 40, 43, 45, 47, 50, 52, 55, 58, 60 degrees). In several embodiments, encapsulated release is linked to the particle (e.g. on a shell or coating layer). In several embodiments, encapsulated release is separate from the particle (e.g. in a separate particle).

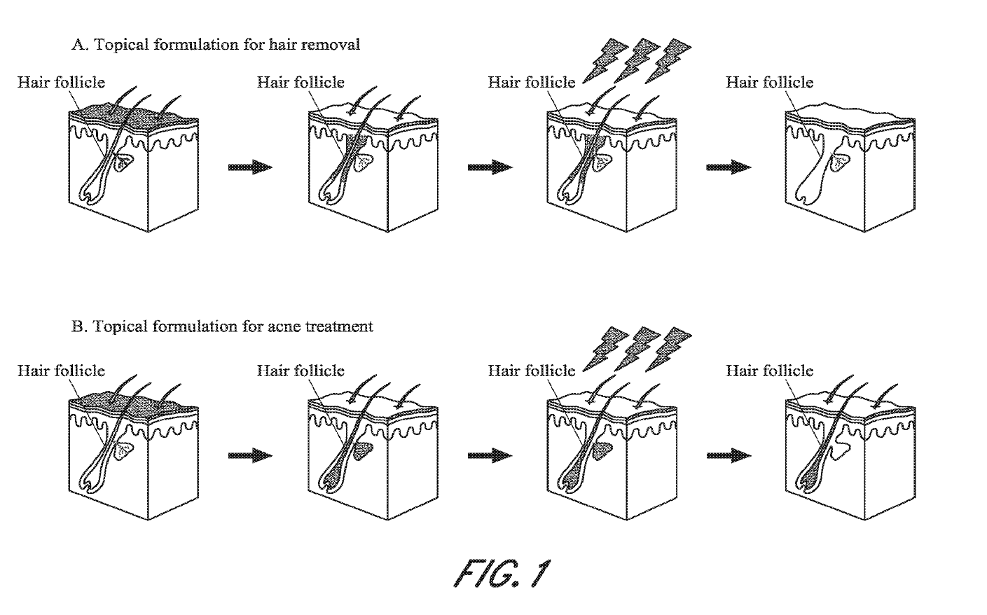

FIG. 1 is illustrative of schematics depicting certain embodiments of the use of compositions, solutions, drugs, and/or formulations for targeting of one or more structures in the skin, such as illustrated for a hair treatment or an acne treatment. Depicted one embodiment in a number of steps at (A) for hair treatment, with a plasmonic nanoparticle formulation (black) at step 1 applied topically to human skin, step 2 delivered deep into the follicle and washed from the skin surface, step 3 irradiated with a clinical laser or a light at a wavelength resonant to the peak absorption wavelength of the plasmonic particle, and step 4 heating of the follicle. In various embodiments, a peak absorption wavelength can be in the infrared range (e.g., 700 nm-1200 nm, 750 nm, 800 nm, 810 nm, 1064 nm, and any ranges or values therein). In some embodiments, the light has a violet wavelength (e.g., 380 nm-450 nm, 390 nm-440 nm, 420 nm, 430 nm, 440 nm, and overlapping ranges therein). In some embodiments, the light has a red wavelength (e.g., 620 nm-750 nm, 630 nm-700 nm, 640 nm-660 nm, 640 nm, 650 nm, and overlapping ranges therein). Depicted in one embodiment in a number of steps at (B) for acne treatment, the plasmonic nanoparticle formulation (black) at step 1 is applied topically to human skin, 2. delivered specifically into the sebaceous gland and washed from the skin surface, 3. irradiated with a clinical laser or a light at a wavelength resonant to the peak absorption wavelength of the plasmonic particle, and 4. shed from the target site where the accumulated sebum and sebum-producing capabilities of the sebaceous gland are destroyed. In some embodiments, the use of agents (such as compositions, solutions, drugs, and/or formulations) for targeting of one or more structures in the skin can comprise both hair treatment and acne treatment. In some embodiments, the use of agents (such as compositions, solutions, drugs, and/or formulations) for targeting of one or more structures a tissue comprises lipolysis.

In several embodiments, the invention relates to using laser or light energy combined with photoactive particles (e.g., nanoparticles) in conjunction with (e.g., before, during/concomitant with, or after) a drug treatment (or other agent) to treat the skin (including tissue under the skin surface) by improving the efficacy and/or facilitating the delivery of the agent (e.g. drug) with plasmonic nanoparticles and/or other photoactive compounds at the target tissues. In various embodiments, the invention is able to selectively target specific tissue, increase tissue permeability, improve kinetics, increase reaction rates, increase chemical interactions for treatment, and/or modify, smooth, and/or resurface the skin (including tissue under the skin surface) of mammals. In some embodiments, mammals are humans. In some embodiments, mammals can be companion animals to humans or commercially beneficial animals to humans. In some embodiments, the improvements are non-linear. In some embodiments, the improvements are exponential. In some embodiments, the improvement is additive with other treatments. In some embodiments, the improvement is synergistic with other treatments. In several embodiments, the agent's efficacy is increased by 10%-100% or 2-10 fold when combined with the nanoparticles as compared to using the agent alone. Efficacy can be used on one or more of the following parameters: enhanced delivery, permeability, localization, absorption, activity or other desired factors. In some embodiments, the agent is an inert compound that becomes activated through a thermal reaction (e.g., cleavage, metabolism, etc.).

As described herein, various embodiments of the invention are used for treating skin tissue with an agent (e.g., drug). In several embodiments of the invention, reduction of microorganisms in the skin, via the photoactive particles (e.g., plasmonic nanoparticles) described herein, include, but is not limited to, inactivation of bacteria, a biofilm, or other microorganisms, reduction in the number, growth, viability, and/or function etc. of bacteria or other microorganisms. In some embodiments, various forms of bacterial infections (e.g., P. acnes, Peptoniphilus, Stenotrophomonas Matltophilia, Staphylococcus, Finegoldia, Pseudomonas, Enterobacter, Serratia, other bacteria, other Gram-positive bacteria, other Gram-negative bacteria, etc.) can be treated. In some embodiments, a treatment can be accomplished by, for example, the heat generated by several of the embodiments described herein and/or the enhanced delivery of drugs and other substances. In some embodiments, light therapies for the prevention and treatment of non-malignant, malignant, melanoma and non-melanoma skin cancers have been focused largely on photodynamic therapy approaches, whereby photosensitive porphyrins are applied to skin and used to localize light, produce reactive oxygen species and destroy cancer cells via toxic radicals. For example, 5-ALA (5-aminolevulinic acid) combined with laser treatment has been FDA-approved for the treatment of non-melanoma skin cancer actinic keratoses, and it is sometimes used for the treatment of widely disseminated, surgically untreatable, or recurrent basal cell carcinomas (BCC). However, this procedure causes some patients to experience photosensitivity, burning, peeling, scarring, hypo-pigmentation and hyperpigmentation and other side effects due to non-specific transdermal uptake of porphyrin molecules. The nanoparticles described herein provide significantly higher photothermal conversion than natural pigments and dyes, enabling laser energy to be focused to specific cells, structures, or components of tissue for selective heating and/or thermomodulation.

In some embodiments, light energy combined with plasmonic nanoparticles and/or other photoactive compounds (which includes photosensitive and photoreactive compounds) in conjunction with (e.g., before, during/concomitant with, or after) a drug treatment (or other agent) may be used for tissue repair, healing acute wounds, healing chronic wounds, healing wounds of surgery, etc. In some embodiments, regenerative repair of skin tissue, prevention and/or treatment of fibrosis is accomplished. Wound repair agents such as those that are anti-inflammatory or that repair or produce new tissue are provided. Agents that remove necrotic tissue or decrease infection can also be provided as wound healing agents. Silver compounds, hyaluronic acid (and salts), anti-microbial compounds, and vitamins are used as wound healing agents in several embodiments.

In some embodiments, plasmonic nanoparticles may be combined with photosensitizers including photodynamic therapy (e.g., 5-ALA) and other chemical compounds sensitive to light. In one embodiment, a photosensitizer is a chemical compound that can be promoted to an excited state upon absorption of light and undergo intersystem crossing with oxygen to produce singlet oxygen. This species rapidly attacks any organic compounds it encounters, thus being highly cytotoxic. In some embodiments, the plasmonic nanoparticles can work in conjunction with a material, or individually, to produce free radicals. In some embodiments, the plasmonic nanoparticles can work in conjunction with a material, or individually, that is highly reactive and produces a therapeutic and/or cosmetic effect. In addition to temperature dependent enhancements of the reaction rate and tissue permeability of photosensitizers, plasmonic nanoparticle can also act as antennas to focus and relay the absorption of light by photosensitizers in close proximity such as from 1 to 100 nm, 1 to 50 nm, 1 to 10 nm, or 5 to 10 nm (e.g. about 1 nm, 5 nm, 10 nm, 20 nm, 25 nm, 30 nm, 40 nm, 50 nm, 60 nm, 70 nm, 75 nm, 80 nm, 90 nm, 100 nm and any ranges therein) from the surface of the plasmonic nanoparticle. A plasmonic nanoparticle with an absorption/scattering cross-section tuned to the excitation wavelength of a photosensitizer compound will increase the rate of promotion of the chemical compound to an excited state producing more singlet oxygen under the same illumination of light. The photosensitizer may be provided in the same composition with a plasmonic nanoparticle, or a plasmonic nanoparticle may be constructed with the photosensitizer compounds bound on or near the surface, achieving an optimal intermolecular distance for excitation

Using the materials and techniques described herein may provide cancer treatments of greater degree, specific targeting, and/or effectiveness than existing methodologies. In certain embodiments, tuned selective ablation of specific target cells, such as Merkel cells or Langerhans cells, can be achieved as described herein. In particular, plasmonic nanoparticles are specifically localized to regions of hair follicles where follicular bulge stem cells arise to form nodular basal cell carcinomas and other carcinomas. Plasmonic nanoparticles may also be delivered to other target cells that cause tumors, for example, the interfollicular epithelium, which include the cell of origin for superficial basal cell carcinomas.

In one embodiment, a composition comprising a cosmetically acceptable carrier and a plurality of photoactive particles (e.g., plasmonic nanoparticles) is provided in an amount effective to induce increase permeability and kinetics in a target tissue region with which the composition is topically contacted, thereby improving the delivery and effectiveness of an agent (such as a drug) with the composition. In some embodiments, the agent may interact with DNA and/or RNA. In some embodiments, a treatment may cleave DNA and/or RNA. In some embodiments, the plasmonic nanoparticles involve gene therapy. In some embodiments, the DNA and/or RNA are the subject's. In some embodiments, the DNA and/or RNA are bacterial or viral. In some embodiments, the composition comprises, or consists essentially of photoactive particles (e.g., plasmonic nanoparticles) that are activated by exposure to energy delivered from a surface plasmon resonance excitation sources (e.g., nonlinear excitation surface plasmon resonance source) to the target tissue region.

As discussed herein, at resonance wavelengths plasmonic nanoparticles can act as antennas, providing a “nonlinear excitation” at peak resonance or, in other words, an enhanced extinction cross section for a given physical cross-section of material when compared to non-plasmonic photoactive materials of the same dimension. Thus, in several embodiments, plasmonic materials are able to pull more energy from delocalized electromagnetic waves surrounding the material at peak resonance than non-plasmonic photoactive material of the same dimension.

In some embodiments, one or more agents (such as drugs, growth factors, healing factors, collagen byproducts, collagen precursors, hyaluronic acid, glucosamine, allantoin, vitamins, oxidants, antioxidants, amino acids, retinoids, retinoid-like compounds, and supplemental minerals among others) are used in combination with nanoparticles or other photoactive compounds. The photoactive compounds includes photosensitive and photoreactive particles and do not necessarily have to be in the nanoparticle size range. In several embodiments, the nanoparticles and photoactive particles are either assembled or unassembled, aggregated or unaggregated, and dimensioned to fit within a target area (such as a pilosebaceous unit, hair follicle, or sebaceous gland). Nanoparticles, as used herein and according to several embodiments, have one dimension (such as a length, width, radius, diameter, thickness, circumference, etc.) in the range from 10 nm to 900 nm, 10 nm to 1000 nm, 10 nm to 300 nm, 50 nm to 500 nm, and overlapping ranges therein. In some embodiments, the ratio of a first dimension to a second dimension is 1:1 to 1:100 (e.g., 1:2, 1:5, 1:10, 1:5, 1:50, etc.). In one embodiment, the nanoparticle is plate shaped having at least one dimension in the range of 30-140 nm (e.g., 35-50 nm, 30-80 nm, 100-130 nm), a sphere having a diameter of 100-200 nm (120-150 nm, 140-160 nm, 125-175 nm), or a rod having a length of 100-900 nm. The photoactive compounds are larger than 1000 nm in all dimensions, according to several embodiments.

In some embodiments, the one or more agents act in an additive or synergistic manner with the nanoparticles or photoactive compounds. In several embodiments, the agents disclosed herein are provided in a therapeutically effective range. In some embodiments, the range is about 0.05-25% (e.g., 0.05-5, or 5-10%, 5-15%, and overlapping ranges therein) per ml of the composition, or % m/m, % m/v, or % v/v of the composition. In some embodiments, glucosamine is provided in an amount sufficient to effectively reduce inflammation. In some embodiments, allantoin is provided in an amount sufficient to heal tissue and improve tissue growth. Agents, in some embodiments, can be steroidal anti-inflammatory drugs, non-steroidal anti-inflammatory drugs, antioxidants, antibiotics, antiviral drugs, antiyeast drugs and antifungal drugs. In various embodiments of the present invention, the vitamins that are used may be vitamin C and/or vitamin E and/or vitamin B and/or vitamin K. In some embodiments, hydrophobic vitamins are used. In some embodiments, hydrophilic vitamins are used. In some embodiments, hydrophilic agents are used. In some embodiments, hydrophobic agents are used. In some embodiments, lipophilic agents are used. In some embodiments, lipophobic agents are used. In some embodiments, agents include copper and/or zinc. The antioxidants can be, for example, vitamin C and/or vitamin E. In one embodiment, the agent comprises any one or more of glycolic acid, sulfur, salicylic acid, and benzoyl peroxide. Skin lightening, whitening, or brightening agents are also provided (including but not limited to anti-melanin agents, hydroquinone, retinoic acid, and steroids). In several embodiments, one or more of the agents described herein are included in the same composition as the photoactive particles. In other embodiments, these agents are provided before, during (concomitant), and/or after treatment with the photoactive particles. In one embodiment, the efficacy of these agents is enhanced when used in combination with the photoactive particles. In some embodiments, the one or more compositions can rejuvenate the skin by having an additive or synergistic effect in the ablation of one or more targeted structures. In some embodiments, the one or more target structures are (e.g., hair follicles, scars, moles, freckles, etc.) on and/or within the skin. In some embodiments, the one or more target structures are tumor, cancer, etc. on and/or within the skin. In some embodiments, the one or more target structures are acne on and/or within the skin.

In several embodiments, the agent has a therapeutic effect, but has no effect on pigment (e.g., does not change pigmentation, is not a colorant or dye, etc.). However, in other embodiments, the agent is designed to affect (increase or decrease) pigmentation. For example, skin-lightening agent(s), in combination with the photoactive particles and the localized heating described herein, are used to treat hyperpigmentation in several embodiments. In several embodiments, the skin lightening agent comprises one or more anti-melanin agents, such as agents that reduce the production or storage of melanin, increase melanin degradation, and/or decrease the melanin transport from melanocytes to keratinocytes. In several embodiments, the skin lightening agent is a tyrosinase inhibitor. In several embodiments, the skin lightening agent comprises at least one, two or more of the following: a retinoid, hydroquinone, mulberry extract, azelaic acid, hydroquinone-β-D-glucoside, ellagic acid, vitamin E, ferulic acid, ascorbic acid, magnesium ascorbyl phosphate, glutathione, arbutin, kojic acid, kojic dipalmitate, lactic acid, glycolic acid, niacinamide, and monobenzone.

In various embodiments, a plasmonic nanoparticle composition is used to selectively target structures in the skin tissue to heat and/or selectively damage tissue with exposure to energy. In various embodiments, heating of tissue with the nanoparticles increases the temperature of a targeted tissue in the vicinity and/or in contact with the nanoparticles by 1, 2, 3, 4, 5, 7, 10, 15, 20, 25, 30, 35, 40, 45, 50 degrees Celsius, or more, and any temperature ranges therein. In various embodiments, irradiation with the composition heats the target tissue to a temperature in the range of about 1 to 80, 1 to 50, 1 to 40, 1 to 30, 1 to 20, 1 to 10 degrees Celsius. In various embodiments, the irradiation with the composition heats the target tissue 1 to 15, 1 to 12, 1 to 7, about 15, 12, 10, 7, 5, 3, or 1 degree Celsius. In various embodiments, irradiation with the composition heats the target tissue to a temperature in the range of about 40 to 45, 40 to 50, 40 to 55, 40 to 60, 40 to 65, 40 to 70, 40 to 75, 40 to 80 or 40 to above 80 Celsius.

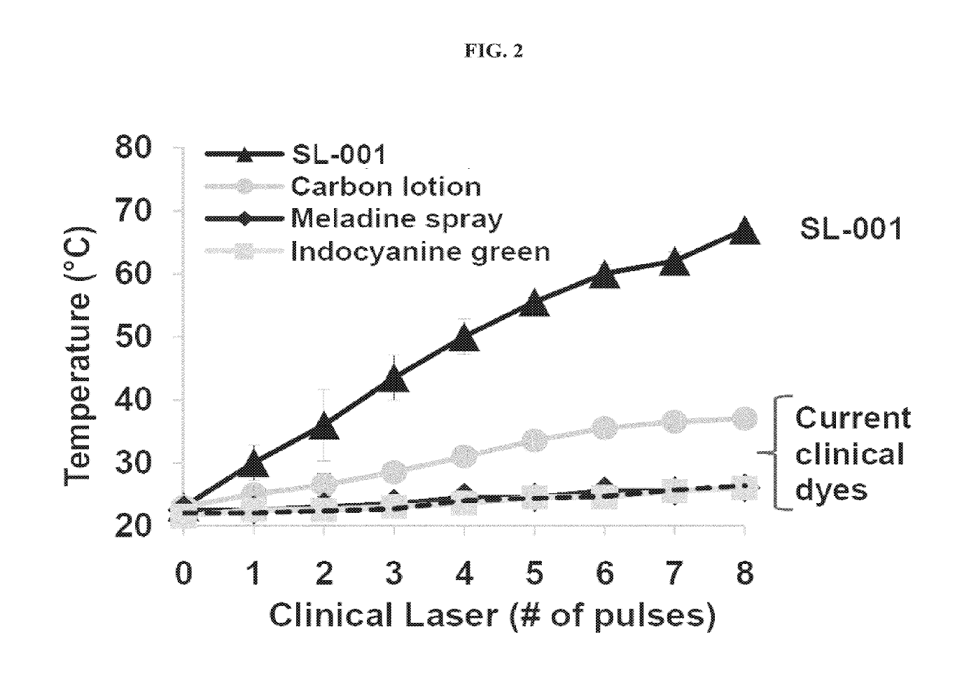

FIG. 2 is illustrative of a temperature profile of certain embodiments of the composition of plasmonic nanoparticles (SL-001, triangles) provided herein compared to certain embodiments of clinical dyes carbon lotion (circles), meladine spray (diamonds), and indocyanine green (squares), after exposure to 1064 nm, 20 J/cm², 55 ms laser pulses. SL-001 and dyes were equally diluted at 1:1000 from clinical concentration (SL-001 1000 O.D., carbon 20-200 mg/ml, meladine 1 mg/ml, ICG 5 mg/ml). n=3, error S.D. of mean.

In some embodiments, described herein are compositions comprising, or consisting essentially of, at least one photoactive particle (e.g., plasmonic nanoparticle) that comprises a metal, metallic composite, metal oxide, metallic salt, electric conductor, electric superconductor, electric semiconductor, dielectric, quantum dot or composite from a combination thereof. In further or additional embodiments, provided herein is a composition wherein a substantial amount of the photoactive particles (e.g., plasmonic particles) present in the composition comprise geometrically-tuned nanostructures. In certain embodiments, provided herein is a composition wherein photoactive particles (e.g., plasmonic particles) comprise any geometric shape currently known or to be created that absorb light and generate plasmon resonance at a desired wavelength, including nanoplates, solid nanoshells, hollow nanoshells, partial nanoshells, nanorods, nanorice, nanospheres, nanofibers, nanowires, nanopyramids, nanoprisms, nanostars, nanocrescents, nanorings, or a combination thereof. In yet additional embodiments, described herein is a composition wherein the photoactive particles (e.g., plasmonic particles) comprises, consists of, or consists essentially of silver, gold, nickel, copper, titanium, silicon, galadium, palladium, platinum, or chromium, as well as including metal alloys, composites, and amalgams.

One or more cosmetically acceptable carriers or other ingredients are used in conjunction with the nanoparticles (or photoactive compounds) and the agent (such as a drug). In some embodiments, provided herein is a composition comprising a cosmetically acceptable carrier that comprises, or consists essentially of, an additive, a colorant, an emulsifier, a fragrance, a humectant, a polymerizable monomer, a stabilizer, a solvent, a copolymer, a polyoxythylene, a polyoxypropylene, a poloxamer derivative, a polysorbate, and/or a surfactant. In one embodiment, provided herein is a composition wherein the surfactant is selected from the group consisting of: sodium laureth 2-sulfate, sodium dodecyl sulfate, ammonium lauryl sulfate, sodium octech-1/deceth-1 sulfate, lipids, proteins, peptides or derivatives thereof. In one embodiment, provided is a composition wherein a surfactant is present in an amount between about 0.1 and about 10.0% weight-to-weight of the carrier. In yet another embodiment, the solvent is selected from the group consisting of water, propylene glycol, alcohol, hydrocarbon, chloroform, acid, base, acetone, diethyl-ether, dimethyl sulfoxide, dimethylformamide, acetonitrile, tetrahydrofuran, dichloromethane, and ethylacetate. In one embodiment, the composition comprises, or consists essentially of, photoactive particles (e.g., plasmonic particles) that have an optical density of at least about 1 O.D. (e.g., 10, 25, 50, 100, 200, 300, 500, 750, 1000, 2000, 5000, 10,000 O.D.) at one or more peak resonance wavelengths at, for example, violet, red, or infrared.

In further or additional embodiments, described herein is a composition wherein photoactive particles (e.g., plasmonic particles) comprise a hydrophilic or aliphatic coating, wherein the coating does not substantially adsorb to skin of a mammalian subject, and wherein the coating comprises polyethylene glycol, silica, silica-oxide, polyvinylpyrrolidone, polystyrene, polyquaternium(s), a protein or a peptide. In one embodiment, the coating is less conductive than a metal portion of the nanoparticle. In some embodiments, the coating is non-conductive. In some embodiments, the coating is semi-conductive. In some embodiments, the nanoparticle is encapsulated in a material. In some embodiments, the nanoparticle encapsulates a material. In some embodiments, the coating comprises a matrix. In some embodiments, the nanoparticle comprises a liposome. In yet an additional embodiment, the thermomodulation comprises damage, ablation, thermoablation, lysis, denaturation, deactivation, activation, induction of inflammation, treatment of inflammation, activation of heat shock proteins, perturbation of cell-signaling or disruption to the cell microenvironment in the target tissue region.

In some embodiments, the target tissue region comprises a pilosebaceous unit, a sebaceous gland, a component of a sebaceous gland, a sebocyte, a component of a sebocyte, sebum, or hair follicle infundibulum. In some embodiments, the target tissue region comprises a dysfunctional and/or infected pilosebaceous unit, a dysfunctional and/or infected sebaceous gland, a component of a dysfunctional and/or infected sebaceous gland, a dysfunctional and/or infected sebocyte, a component of a dysfunctional and/or infected sebocyte, sebum from a dysfunctional and/or infected sebaceous gland, or a dysfunctional and/or infected hair follicle infundibulum. In further embodiments, the target tissue region comprises a bulge, a bulb, a stem cell, a stem cell niche, a dermal papilla, a cortex, a cuticle, a hair sheath, a medulla, an arrector pili muscle, a Huxley layer, a Henle layer or an apocrine gland.

In one embodiment, described herein are methods of performing targeted enhancement of permeability and/or kinetics of biochemical process in or near tissue. For example, in one embodiment, provided is a method for enhancing a treatment, comprising the steps of applying a drug to a skin surface, topically administering a composition of photoactive particles (e.g., plasmonic particles), providing penetration means to redistribute the plasmonic particles from the skin surface to a component of dermal tissue; and irradiating the skin surface by light.

In various embodiments, the light source is a device (e.g., lamp, bulb, laser, LED, etc.). In some embodiments, the light source is natural (e.g., sun light). In some embodiments, the light source can be ultraviolet (UV). In various embodiments a visible spectrum light source, infrared, and/or near-infrared light source is used. In various embodiments, a visible spectrum light source (e.g., violet 380 nm-450 nm wavelength, blue 450 nm-495 nm wavelength, green 495 nm-570 nm wavelength, yellow 570 nm-590 nm wavelength, orange 590 nm-620 nm wavelength, red 620 nm-750 nm wavelength, 380 nm-750 nm, 350 nm-700 nm, 400 nm-450 nm, 410 nm-440 nm, 440 nm, 620 nm-700 nm, 630 nm-660 nm, 640 nm, and ranges and values therein) is used. In some embodiments, the light has a violet wavelength (e.g., 380 nm-450 nm, 390 nm-440 nm, 420 nm, 430 nm, 440 nm, and overlapping ranges therein). In some embodiments, the light has a red wavelength (e.g., 620 nm-750 nm, 630 nm-700 nm, 640 nm-660 nm, 640 nm, 650 nm, and overlapping ranges therein). In various embodiments, an infrared light source (e.g., 700 nm-1 mm wavelengths, 700 nm-1200 nm, 750 nm-1200 nm, 700 nm, 755 nm, 800 nm, 810 nm, 1064 nm, 1200 nm, etc. and overlapping ranges therein) is used. In various embodiments, a near-infrared light source (e.g., 700 700 nm-1200 nm, 750 nm-1200 nm, 700 nm, 755 nm, 800 nm, 810 nm, 1064 nm, 1200 nm, etc. and overlapping ranges therein) is used. In various embodiments, the irradiation is provided continuously. In various embodiments, the irradiation is pulsed. In some embodiments, the enhancements may be enabled with continuous light exposure and/or pulsing. Pulsing may offer additional benefits by causing much higher local temperatures in skin microstructures (e.g. pilosebaceous units) during short pulses that provide non-linear increases in permeability and reaction kinetics at the site of action. With pulsing this can be done without raising the bulk surrounding tissue much, or at all. In various embodiments, the pulses can be provided in 0.1, 0.5, 1, 2, 3, 4, 5, 10, 15, 20, 30, 40, 50, 75, 100, 200, 500, and 1000 ms, or any range therein. In some embodiments, the pulsed irradiation is provided in a range of 5-30 ms, 1-50 ms, 5-10 ms, and/or 20-100 ms. The time period may be in the range of 1 femtosecond to about 1 second (e.g., 100 microsecond to 1000 microseconds, 1 millisecond to 10 millisecond, 10 millisecond to 100 millisecond, 100 millisecond to 500 millisecond).

In some embodiments, provided is a method wherein the light source comprises excitation of mercury, xenon, deuterium, or a metal-halide, phosphorescence, incandescence, luminescence, light emitting diode, or sunlight. In still further embodiments, provided is a method wherein the irradiation comprises light having a wavelength of light between about 200 nm and about 10,000 nm (e.g., 700 nm to 1,200 nm, 600 nm to 1,500 nm, 500 nm to 2,000 nm), a fluence of about 0.1 to about 100 joules/cm² (e.g., 1 to 60 joules/cm², 1 to 5 joules/cm², 5 to 50 joules/cm², 10 to 30 joules/cm²), a pulse width of about 1 femtosecond to about 1 second (e.g., 100 microsecond to 500 millisecond, 100 microsecond to 1000 microseconds, 1 millisecond to 10 millisecond, 10 millisecond to 100 millisecond, 100 millisecond to 500 millisecond), and a repetition frequency of about 1 Hz to about 1 THz (e.g., 1 Hz to 10 Hz, 1 Hz to 1 MHz, 1 Hz to 1 GHz).

In various embodiments, a method of topically delivering a composition is provided, including delivering one or more agents (e.g., a drug) and photoactive particles (e.g., nanoparticles) to a target tissue under a skin surface, either simultaneously or sequentially. The method includes applying the composition to a skin surface, and distributing the composition from the skin surface to a target tissue under the skin surface (optionally using massage, ultrasound or pressure to do so). For embodiments to process hair (such as hair growth, reduction in hair growth, hair coloring, hair straightening, or hair curling), the composition(s) are applied to the hair and/or scalp. Hair removal (e.g., removing undesired hair on the head (e.g., face, eyebrows, moustache, beard, scalp), underarms, back, legs or other body part) is also provided. In some embodiments, the hair removal is semi-permanent (such as months or years) or permanent. In one embodiment, future hair growth is reduced by more than 25, 50 or 75%. In some embodiments, a first composition comprises a plurality of plasmonic nanoparticles and a second composition includes one or more agents. In these embodiments, the nanoparticle composition can be applied before, after, or simultaneously with the agent composition. Kits are provided in some embodiments that comprise either separate containers of the agent and the nanoparticles or containers comprising the two combined together. A hand-held energy source is provided in some embodiments for home use by a consumer.

In one embodiment, the plasmonic nanoparticles comprise a conductive metal portion. In one embodiment, the conductive metal portion comprises at least one of gold or silver. In one embodiment, the plasmonic nanoparticles have a size in a range of 10 to 1,000 nm (e.g., 10 nm to 300 nm, 10 nm to 100 nm, 50 nm to 150 nm, 100 nm to 200 nm, 100 nm to 250 nm, 100 nm to 300 nm, 100 nm to 500 nm, 150 nm to 250 nm, 100 to 700 nm, 100 nm to 900 nm, and any ranges therein). In one embodiment, the plasmonic nanoparticles comprise a coating that coats the conductive metal portion, wherein the coating facilitates selective removal from the skin surface. In one embodiment, the coating is less conductive than the metal portion. In one embodiment, the coating comprises at least one of silica or polyethylene glycol (PEG). In one embodiment, the plasmonic nanoparticles have a concentration of 10⁹ to 10²³ particles per volume of the composition (e.g., preferably 10⁹, 10¹⁰, 10¹¹, 10¹², 10¹³, 10¹⁴, 10¹⁵, 10¹⁶ per ml of the composition, and any concentrations and ranges therein such as 10¹⁰-10¹⁶, 10¹¹-10¹⁵, 10⁹-10¹⁶, 10¹⁰-10¹³ per ml of the composition), wherein the concentration is sufficient to, after exposure to irradiation, induce thermal damage in a target tissue. In one embodiment the method includes selectively removing the composition from the skin surface, while leaving the composition localized within the target tissue. In one embodiment the method includes irradiating the composition with a light source thereby inducing a plurality of surface plasmons in the plasmonic nanoparticles. In one embodiment, the plurality of surface plasmons generates localized heat in the target tissue, and such selective heating of the target tissue provides advantages when combined with drug activities. The nanoparticles may be assembled or unassembled. The nanoparticles of the invention can generally contain a collection of unassembled nanoparticles. By “unassembled” nanoparticles it is meant that nanoparticles in such a collection are not bound to each other through a physical force or chemical bond either directly (particle-particle) or indirectly through some intermediary (e.g. particle-cell-particle, particle-protein-particle, particle-analyte-particle). In addition, the nanoparticles may be aggregated or unaggregated.

In several embodiments, the methods can be performed in any order, with any step repeated one or more times. In some embodiments of the methods, the contacting and/or delivering steps can be repeated 1, 2, 3, 4, 5, 10 or more times. In several embodiments, the methods for treating the target regions are repeated one or more times on one or more additional target regions. For example, the procedure may be performed/repeated 1-10 times (e.g., 2, 3, 4, 5, 10 or more times). A single target region may be treated, or alternatively, multiple target regions may be treated sequentially or simultaneously.

In several embodiments, the photoactive material comprises carbon. In several embodiments, the photoactive material comprises graphite. In several embodiments, the photoactive material comprises a plasmonic nanoparticle. In several embodiments, the photoactive material comprises a silver plasmonic nanoparticle. In several embodiments, the photoactive material comprises a silica-coated silver plasmonic nanoparticle. In several embodiments, the photoactive material is present at a concentration of from about 0.01% to about 10% volume to volume ratio, or greater than 10% volume to volume ratio (e.g., 0.01%-0.1%, 0.1%-1%, 1%-10%. In several embodiments, the photoactive material does not substantially penetrate the epidermal surface. In several embodiments, the energy comprises a spot diameter anywhere in the range of about 0.5 mm to about 20 mm (e.g., about 0.5-10, 1-5, 3-15 mm) at the epidermal surface.

In some embodiments, the photoactive particles comprise a silver or gold nanoplate, nanoshell or nanorod. The nanoplate and nanorod can be solid metal (with or without a non-metal coating to, for example, facilitate removal from the target) or can be partially metal, such has having a metal core (with a non-metal outer layer) or a metal outer layer (with a non-metal core). In one embodiment, a non-metal layer is provided between two or more metal layers. The nanoparticles, according to some embodiments, have a first dimension in the range of 50 nm-350 nm, a second dimension in the range of 10 nm-500 nm, and are sized to fit within the target area at the desired concentration.

In some embodiments, the invention comprises a kit for treating the skin. In some embodiments, the invention comprises a kit for treating a tissue. In some embodiments, the invention comprises a kit for treating the mouth, nose, eye, mucous membranes, gums, digestive tract, respiratory tract, circulatory tract, urinary tract, an organ, a bone, a fingernail, a toe nail, and/or hair. In some embodiments, a transdermal patch is used. In some embodiments, the invention comprises a kit for treatment with a pill. In some embodiments, the invention comprises a kit for treatment with an energy source that is applied the skin. The kit includes some or all of the following, in several embodiments: photoactive particles (such as nanoparticles and/or chromophores) together with or separately from the agent, means for delivering the composition to the skin (e.g., to atrophic regions or other target regions), a light source, and instructions for use.

In some embodiments a means of removing the photoactive particles from the skin or modifying the distribution of the composition on the skin is provided. Accordingly, in several embodiments, after the composition of nanoparticles is applied to the skin or target region, the excess is wiped off or otherwise removed to, for example, facilitate localized heating. If the agent is provided in a composition separate from the nanoparticles, such composition may optionally be removed as well.

An object of the subject matter described herein is to provide compositions, methods and systems for noninvasive and minimally-invasive treatment of skin and underlying tissues, or other accessible tissue spaces with the use of photoactive compounds (including but not limited to photoactive particles such as nanoparticles, plasmonic nanoparticles, etc.). In some embodiments, the invention describes the development and utilization of compositions containing photoactive materials (e.g., nanoparticles and other materials) for the treatment of small target regions of skin. In one embodiment, such compositions are generally applied topically, through an apparatus that provides the composition in a form suitable for contact with and retention at a target region of skin in a manner that encompasses irradiating the skin with light (e.g., electromagnetic radiation) having a wavelength sufficient to heat the target region of skin and increase the permeability of skin and kinetics of a supplemental drug or other compound.

In various embodiments, the treatment includes, but is not limited to, hair removal, hair growth and regrowth, and skin rejuvenation or resurfacing, acne removal or reduction, wrinkle reduction, sagging skin reduction, pore reduction, reduction and/or mitigating of the appearance of aging, reduction and/or mitigating of the appearance of fatigue, skin lightening, ablation of cellulite and other dermal lipid depositions, lipolysis, wart and fungus removal, thinning or removal of scars including hypertrophic scars and keloids, abnormal pigmentation (such as port wine stains), tattoo removal, and skin inconsistencies (e.g. in texture, color, tone, elasticity, hydration, and including sun spots, age spots, freckles, and other inconsistencies). Other therapeutic or preventative methods include but are not limited to treatment of hyperhidrosis, anhidrosis, Frey's Syndrome (gustatory sweating), Homer's Syndrome, and Ross Syndrome, actinic keratosis, sebhorreic keratosis, keratosis follicularis, dermatitis, vitiligo, pityriasis, psoriasis, lichen planus, eczema, alopecia, psoriasis, malignant or non-malignant skin tumors, onychomycosis, sebhorreic dermatitis, atopic dermatitis, contact dermatitis, herpes simplex, Human papillomavirus (HPV), and dermatophytosis.

In several embodiments, the agents (in combination with the photoactive particles disclosed herein) are used to treat psoriasis, eczema and/or dermatitis. In some embodiments, the agent comprises a topical cytokine (e.g., interleukin) inhibitor. Agents, in some embodiments, include inhibitors to one or more of the following compounds: TNF, EGF, and interleukins (IL-1, IL-2, IL-3, IL-17, etc.). In some embodiments, topical calcinuerin inhibitors are used for the treatment of psoriasis, eczema and/or dermatitis. Calcineurin phosphatase inhibitors are used as agents in one embodiment. Also provided in some embodiments are agents that cause the inhibition of the activation of T cells and/or the inhibition of the production of proinflammatory cytokines (e.g., IL-2, TNF-α, IFN-γ etc.). Pimecrolimus and tacrolimus are provided as agents in some embodiments to treat psoriasis, eczema and/or dermatitis. Janus kinase (JAK) inhibitors are used as agents in other embodiments. Topical steroids are provided as agents in several embodiments. The agents to treat psoriasis, eczema and/or dermatitis (and other conditions described herein) are used, in some embodiments, in the range of 0.05-5% (e.g., 0.5-3%, 1-5%, 0.05-2%, etc.) per ml of the composition or % m/m, % m/v, or % v/v of the composition to achieve a therapeutic effect. The agents, when used with the photoactive particles (such as plasmonic nanoparticles) described herein, result in, for example, enhanced penetration and efficacy of the agents in several embodiments.

In some embodiments, the agent is an aminolevulinic compound (e.g, δ-aminolevulinic acid) and is combined with the photoactive particles described herein to treat actinic keratosis and acne. In some embodiments, the aminolevulinic compound is provided in the range of 1-25% (e.g., 2-10%, 10-20%, 10-25%, etc.) per ml of the composition or % m/m, % m/v, or % v/v of the composition to achieve a therapeutic effect.

In some embodiments, the agent is an aminolevulinic compound (e.g, δ-aminolevulinic acid) and is combined with the photoactive particles described herein to treat actinic keratosis, acne or other applicable condition. In some embodiments, the aminolevulinic compound is provided in the range of 1-25% (e.g., 2-10%, 10-20%, 10-25%, etc.) per ml of the composition or % m/m, % m/v, or % v/v of the composition to achieve a therapeutic effect.

In some embodiments, the agent is a keratolytic compound. In one embodiment, the keratolytic compound treats warts and other conditions (such as lesions in which the epidermis produces excess skin). Resorcinol, sulfur, urea, lactic acid, salicylic acid, benzoyl peroxide, and/or allantoin are used in several embodiments. Keratolytics may be used alone or in combination with other types of agents to treat acne, keratosis, eczema, psoriasis or conditions where softening of keratin is beneficial. Hyperpigmentation is treated in one embodiment. When combined with the photoactive particles described herein, the keratolytic is provided in the range of 0.05-25% (e.g., 0.05-5%, 1-10%, 10-25%, etc.) per ml of the composition or % m/m, % m/v, or % v/v of the composition to achieve a therapeutic effect.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described herein. The materials, methods, and examples are illustrative only and not intended to be limiting. Other features of the disclosure are apparent from the following detailed description and the claims.

In some embodiments, the compositions of the instant disclosure are topically administered. Provided herein are means to redistribute plasmonic particles and other compositions described herein from the skin surface to a component of dermal tissue including a hair follicle, a component of a hair follicle, a follicle infundibulum, a sebaceous gland, or a component of a target tissue using ultrasound, high frequency ultrasound, low frequency ultrasound, massage, iontophoresis, use of continuous and/or intermittent electrical pulses, high pressure air flow, high pressure liquid flow, vacuum, pre-treatment with Fractionated photothermolysis laser or dermabrasion, bubble formation, liquid microstreaming, or a combination thereof.

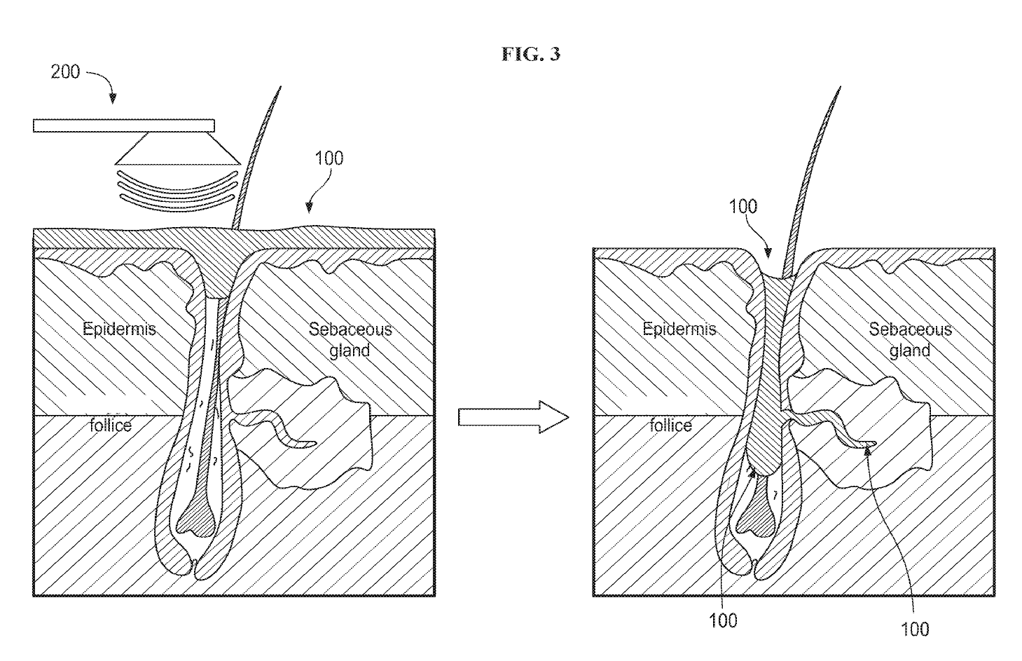

In one embodiment, illustrated at FIG. 3, a delivery device 200 is used to assist in the delivery, distribution, to redistribute, penetrate, drive, disperse, direct, and/or enhance movement of a composition 100 to a target location. In some embodiments, the delivery device 200 is a light device and/or a mechanical vibration device. In some embodiments, the delivery device 200 is a mechanical vibration device configured for mechanical mixing. In one embodiment, a mechanical vibration device vibrates at frequencies of less than 1 kHz, 1 Hz-900 Hz, 5-500 Hz, 10-100 Hz, 1-80 Hz, 50-250 Hz, and any frequencies therein. In some embodiments a mechanical vibration device vibrates, laterally, longitudinally, or radially. In several embodiments of the invention, low frequency ultrasound can be applied at frequencies of 1 kHz to 500 kHz, e.g., 1 kHz-100 kHz, 5 kHz-45 kHz, 20 kHz-50 kHz, 30 kHz-40 kHz, 30 kHz, 40 kHz, and any ranges or frequencies therein.) In several embodiments of the invention, massage (e.g., hand massage, vibration, mechanical vibration) can be applied at frequencies of less than 1 kHz, 1 Hz-900 Hz, 5-500 Hz, 10-100 Hz, 1-80 Hz, 50-250 Hz, and any frequencies therein.

In some embodiments, the delivery device 200 is configured to deliver a composition 100 to a depth of 1-100 microns, 1-1000 microns, 1-1500 microns, 1-2000 microns, 1-3000 microns, 1-4000 microns, and/or 1-5000 microns. In some embodiments, the delivery device 200 is configured to deliver a composition 100 to a depth of 1000-1500 microns. In some embodiments, the delivery device 200 is configured to deliver a composition 100 to a depth of 1000 microns. In some embodiments, the delivery device 200 is configured to deliver a composition 100 to a depth of 1500 microns. In some embodiments, the delivery device 200 is configured to deliver a composition 100 to a depth of 2000 microns. In some embodiments, the delivery device 200 is configured to deliver a composition 100 to a depth of 2500 microns. In some embodiments, the nanoparticles described herein are formulated to penetrate much deeper—up to several centimeters, or into the panniculus adiposus (hypodermis) layer of subcutaneous tissue. For example, the compositions can be administered by use of a sponge applicator, cloth applicator, spray, aerosol, vacuum suction, high pressure air flow, high pressure liquid flow direct contact by hand ultrasound and other sonic forces, mechanical vibrations, physical manipulation, hair shaft manipulation (including pulling, massaging), physical force, thermal manipulation, or other treatments. Nanoparticle composition treatments are performed alone, in combination, sequentially or repeated 1-10 times.

In various embodiments, removing nanoparticles localized on the surface of the skin may be performed by contacting the skin with acetone, alcohol, water, air, a debriding agent, or wax. Alternatively, physical debridement may be performed. Alternatively, one can perform a reduction of the plasmonic or other compound.

In various embodiments, agents for tissue engineering are activated with energy from photoactive compounds (such as plasmonic nanoparticles). In some embodiments, one, two, three, or more polymers form a network for tissue repair or tissue bulking via crosslinking of polymers. In one embodiment, hydrophilic polymers are used. In one embodiment, hydrophobic polymers are used. In one embodiment, polymer crosslinking is activated via application of energy to plasmonic nanoparticles. Such tissue engineering materials can be used for cosmetic, aesthetic procedures, and/or reconstructive surgery. In some embodiments, a polymer solution comprises drugs, hormones, proteins, nucleic acid molecules, polysaccharides, synthetic organic and/or inorganic molecules.

In various embodiments, an agent comprising a filler, such as a dermal filler (e.g., collagen, hyaluronic acid, etc.) is expanded, solidified, cured, or otherwise activated upon application of heat from photoactive compounds (such as plasmonic nanoparticles). In some embodiments, the addition of nanoparticles that generate localized heating upon exposure to light facilitate the filler activation process in a more efficient manner (e.g., reduced time, reduced risk that the filler migrates, and/or more controllability and precision, etc.). The improved activation of fillers (e.g., with lip plumping, skin enhancement, wrinkle removal, scar reduction, etc.) in cosmetic applications include delivery of the agent to a target site independent of, or in conjunction with, agent injection to target site.

Non-cosmetic applications similar to filler are also provided. For example, in various embodiments, an agent comprising a material (e.g., orthopedic composition, non-cosmetic filler, cement, polymer, polymethyl methacrylate, powder, stabilizer, inhibitor) is expanded, solidified, cured, or otherwise activated upon application of heat from photoactive compounds (such as plasmonic nanoparticles). The addition of nanoparticles that generate localized heating upon exposure to light facilitate the efficient use of these agents (e.g., reduced time, reduced risk that the filler migrates, and/or more controllability and precision, etc.).

In various embodiments, a transdermal patch with an agent (e.g., therapeutic material) and plasmonic nanoparticles is applied to a tissue surface (e.g., a skin surface). Energy (e.g., light, laser, ultraviolet, violet wavelengths, red wavelengths, infrared, etc.) is applied to the transdermal patch, thereby activating the drug and enhancing the delivery of the agent to the tissue surface. In one embodiment, a transdermal patch provides for controlled release of the agent into the tissue surface at a controllable rate, as activated by energy delivered to the plasmonic nanoparticles. The transdermal patch is adapted to (e.g., configured to) provide a selective, controlled level of drugs to the tissue at the tissue surface and in some embodiment, below the tissue surface via pores, hair follicles, and/or increased skin permeability. In one embodiment, a transdermal patch is applied to the skin and activated with the plasmonic nanoparticles. The transdermal patch may be left in place to provide multi-day dosing that is convenient for the patient. In one embodiment, the rate of drug delivery is controlled by the activation of the plasmonic nanoparticles. In one embodiment, the amount of adhesion in a transdermal patch is controlled by the activation of plasmonic nanoparticles.

In various embodiments, the agent is a hair product that is applied to the hair (e.g., root, shaft, follicle, sheath, strand, etc.). In various embodiments, localized heating from the nanoparticles causes the hair product to penetrate the hair more deeply, more quickly, and/or more effectively. In other embodiments, use of the hair product without localized heating via the nanoparticles can cause fumes and/or irritants. In some embodiments, use of nanoparticles with the hair product reduces the production of fumes and/or irritants. In one embodiment, the hair product is a hair growth accelerator (e.g., minoxidil). In one embodiment, the hair product is a hair growth inhibitor or hair removal agent (e.g., calcium hydroxide, lime, sodium hydroxide, lye, potassium thiogycolate, etc.). Epilation agents are provided in several embodiments. Through the combination of the photoactive particles and the agents described herein, removal of light or unpigmented hairs and/or removal of darker hair on darker skin are provided in multiple embodiments. In one embodiment, the hair product is a hair dye (e.g., pigments, hydrogen peroxide, pyrogallol, plant compounds, metallic compounds, metal oxides, amino dyes, and modifiers). In various embodiments, the hair product is a hair curling agent (e.g., oil, alcohol, humectant, polymer, cationic polymer, and/or perm agent etc.). In various embodiments, the hair product is a hair smoothing or straightening agent (e.g., relaxers, alkali, keratin, methylene glycol, and/or formaldehyde, formaldehyde substitutes, or other aldehydes). In several embodiments, the use of the nanoparticles or photoactive particles described herein (i) reduce the time for processing and/or (ii) reduce the amount of chemical needed to treat the hair, in each case reducing damage to the hair and/or exposure to any chemical fumes. In some embodiments, heating via irradiation of plasmonic nanoparticles can be used to heat hair in more rapid and/or localized fashion, thereby reducing the amount of heat (and potential damage) delivered during the dying/curling/straightening process (e.g., via hair irons, hair curlers, hair dryers, and other hair heating and styling approaches).

Amount of energy provided. In some embodiments, skin is irradiated at a fluence of 1-100 Joules per cm² with light wavelengths of about 200 nm to 1500 nm, 350 nm to 750 nm, 750 nm to 1200 nm (e.g., 440 nm, 640 nm, 750 nm, 810 nm, 1064 nm), or other wavelengths, particularly in the range of visible or infrared light. Various repetition rates are used from continuous to pulsed, e.g., at 1-10 Hz, 10-100 Hz, 100-1000 Hz. While some energy is reflected, it is an advantage of the subject matter described herein is that a substantial amount of energy is absorbed by particles, with a lesser amount absorbed by skin. Nanoparticles are delivered to the hair follicle, infundibulum, or sebaceous gland at concentration sufficient to absorb, e.g., 1.1-100× more energy than other components of the skin of similar volume. This is achieved in some embodiments by having a concentration of particles in the hair follicle with absorbance at the laser peak of 1.1-100× relative to other skin components of similar volume.

To enable tunable and selective heating of target skin structures (e.g., sebaceous glands, infundibulum, hair follicles) and/or encapsulation materials, some embodiments of light-absorbing nanoparticles are utilized in conjunction with a laser or other excitation source (lamp, bulb, LED, sunlight, UV, IR, etc.) of the appropriate wavelength. The light may be applied continuously or in pulses with a single or multiple pulses of light. The intensity of heating and distance over which heating or photothermal damage will occur are controlled by the intensity and duration of light exposure. In some embodiments, pulsed lighting is utilized in order to provide localized thermal heating or tissue destruction. In some such embodiments, pulses of varying durations are provided to localize thermal damage regions to within 0.05, 0.1, 0.5, 1, 2, 5, 10, 20, 30, 50, 75, 100, 200, 300, 500, 1000 microns of the particles. Pulses are at least femtoseconds, picoseconds, microseconds, or milliseconds in duration. In some embodiments, the peak temperature realized in tissue from nanoparticle heating is at least 5, 10, 15, 20, 25, 30, 40, 50, 60, 70, 80, 90, 100, 200, 300, or 500 degrees Celsius. In some embodiments that utilize pulsed heating, high peak temperatures are realized locally within the hair shaft without raising the macroscopic tissue temperature more than 0.1, 0.5, 1, 2, 3, 4, 5, 7, 9, 12, 15, or 20 degrees Celsius. In some embodiments short pulses (100 nanoseconds-1000 microseconds) are used to drive very high transient heat gradients in and around the target skin structure (e.g., sebaceous gland and/or hair follicle) from embedded particles to localize damage in close proximity to particle location. In other embodiments, longer pulse lengths (1-10 ms, 1-100 ms, or 1-500 ms) are used to drive heat gradients further from the target structure to localize thermal energy to stem cells in the bulge region or other components greater than 100 um away from the localized particles. Fluences of 1-10 Joules per cm², 1-20 Joules per cm², or 1-30 Joules per cm² are generally sufficient to thermally ablate follicles that have high particle concentrations and thus higher absorbance than skin (e.g., 1.1-100 times per volume absorbance of skin). These fluences are often lower than what is currently employed (e.g., Diode: 25-40 J/cm², Alexandrite: 20 J/cm², Nd:YAG: 30-60 J/cm²) and lead to less damage to non-follicular regions, and potentially less pain. Lower fluences can be provided for heating (at sub-ablative levels) of tissue. In several embodiments (e.g., adapted for home use by a consumer), hand-held devices are provided (including but not limited to diode lasers, home pulsed light, intense pulsed light, etc.). In several embodiments (e.g., adapted for home use by a consumer), hand-held devices having an output wavelength in the range of violet, red, infrared, 400-700 nm, 750-1050 nm, or 1200-1600 nm and overlapping ranges therein are provided. In some embodiments (e.g., adapted for home use by a consumer), hand-held devices having an output in the range of 4-15 mJ per pulse are provided. In several embodiments (e.g., adapted for home use by a consumer), hand-held devices include one or more (or all of the following specification): wavelengths of light from 400 to 1200 nm (e.g., 440 nm, 640 nm, 750 nm, 800 nm, 1064 nm, etc.), a maximum energy density of 1-20 J/cm² (e.g., 3-6 J/cm²), a pulse rate of one pulse every 1, 2, 3, 5, 6, 10 seconds (and any values or ranges therein), 2-6 seconds, and a spot size of 10-20 mm x 10-30 mm. In some embodiments, rather than a hand-held device, a covering, mask or other wearable is provided to provide the light. For example, a mask or other wearable can be provided with one or multiple light sources to active the photactive particles. Such wearables may be particularly beneficial for the at-home consumer. In various embodiments, a home use device includes one or more safety mechanism to ensure that the device is aimed at skin and or detecting the presence of plasmonic particles of a discrete dimension and/or area of coverage in order to activate. In some embodiments, a device includes sensors, cameras, detection devices, or other mechanisms. In various embodiments, a plasmonic nanoparticle composition is used to selectively target structures in the skin tissue to increase drug reaction and action kinetics of the tissue at the specifically localized target structure. The faster drug reactions can improve the speed and efficiency with which a drug acts at the target structure. In one embodiment, heating of the specifically targeted structure through irradiation of the nanoparticles heats the tissue at and around the nanoparticles to increase a reaction rate based on the temperature. In one embodiment, the reaction rate for drugs or other agents is related to temperature based on the Arrhenius equation, shown below at equation (1):

k=Ae^{-E}^{_{α}}^{/(}RT) (1)

Arrhenius' equation at (1) gives the dependence of the rate constant k of a chemical reaction on the absolute temperature T (in kelvin), where A is the pre-exponential factor (or simply the prefactor), E_{α} is the activation energy (with energy units uses energy per mole), and R is the universal gas constant. Alternatively, the Arrhenius equation may be expressed as equation (2):

k=Ae^{-E}^{_{α}}^{/(k}^{_{B}}^{T)} (2)

Arrhenius' equation at (2) gives with energy units of E_{α}: (with energy per molecule directly, which is common in physics). The different units in equation (1) and (2) are accounted for in using either R=gas constant or the Boltzmann constant k_{B} as the multiplier of temperature T. Thus, in one embodiment, using nanoparticles to selectively target skin structures to increase the temperature of the target skin structure results in a non-linear increase in reaction kinetics. In one embodiment, using nanoparticles to selectively target skin structures to increase the temperature of the target skin structure results in an exponential increase in reaction kinetics.

In various embodiments, a plasmonic nanoparticle composition is used to selectively target structures in the skin tissue to improve permeability of an agent (e.g. drug) active in tissue localized to the selective heat that is applied at the specifically localized target structure Improved permeability can increase the speed and efficiency with which an agent (e.g. drug) acts at the target structure. In one embodiment, heating of the specifically targeted structure through irradiation of the nanoparticles heats the tissue at and around the nanoparticles to increase a reaction rate based on the temperature under the Arrhenius equation. For example, experiments on the relationship between temperature and permeability are discussed in “Effect of Experimental Temperature on the Permeation of Model Diffusants Across Porcine Buccal Mucosa” AAPS Pharm Sci Tech. 2011 June; 12(2)579 authored by Upendra Dilip Kulkarni, Ravichandran Mahalingam, Xiaoling Li, Indiran Pather, and Bhaskara Jasti, which is incorporated by reference in its entirety herein. This publication reports “The influence of experimental temperature on the permeability of model diffusants across porcine buccal mucosa was investigated in vitro. The permeability increased significantly as the experimental temperature was increased in increments of approximately 7° C. It was observed that the apparent permeability and temperature were related by an exponential relationship that conformed to the Arrhenius equation.” In one embodiment, topical temperature increases of the skin result in increased permeability of the skin.

In some embodiments, the kinetic and/or permeability enhancements take place in the absence of any tissue damage or ablation by irradiation of the nanoparticles. In some embodiments, selective tissue damage incurred with the nanoparticles may be used as an enabling, synergistic feature for combining with drug or other therapy. For example, in one embodiment, the wound healing response activated by selective damage may be supplemented by a drug or other therapy. For example, in one embodiment, after damaging a pilosebacous unit, a concomitant therapy that promotes regrowth of terminal hairs is provided using for example the regrowth technology described in U.S. Pat. No. 8,871,711, which is incorporated by reference in its entirety herein.

In various embodiments, the irradiation is provided continuously. In various embodiments, the irradiation is pulsed. In some embodiments, the enhancements may be enabled with continuous light exposure and/or pulsing. Pulsing may offer additional benefits by causing much higher local temperatures in skin microstructures (e.g. pilosebaceous units) during short pulses that provide non-linear increases in permeability and reaction kinetics at the site of action. With pulsing this can be done without raising the bulk surrounding tissue much, or at all. The Arrhenius equation suggests that the same fluence delivered to the skin via pulsing the power over a period of time versus continuous power over the same time would increase permeability and reaction kinetics in the pulsing scenario more than the continuous power scenario.

### Examples

The following examples illustrate various non-limiting embodiments. Although certain drugs are disclosed, non-drug agents may also be used. Additionally, the terms “formulation” and “composition” can be used interchangeably.

The agents disclosed below will be provided in a therapeutically effective range. In some embodiments, the range will be about 0.05-25% (e.g., 0.05-5, 1-3, 2-5, 4-9%, 5-25% and overlapping ranges therein) per ml of the composition, or % m/m, % m/v, or % v/v of the composition.

### Example 1: Composition of Plasmonic Nanoparticles and Drug

In various embodiments, plasmonic nanoparticles, including nanoplates, nanorods, hollow nanoshells, silicon nanoshells, nanorice, nanowires, nanopyramids, nanoprisms, and/or other configurations described herein, will be generated in size ranges in which at least one dimension is in the range from 1 to 1000 nm (e.g., 10-900, 100-700, 10-300, 50-200, 100-300, 100-200, 1-150 nm and any range or value therein) under conditions such that surface properties that facilitate deep follicular penetration. For example, in one embodiment, the nanoparticle will be a rectangular nanoplate with a first dimension in the range of 50-120 nm (e.g., 100 nm) and a second dimension in the range of 5-50 nm (e.g., 10 nm). Penetration into follicular openings of 10-200 um (e.g., 10-150, 20-100, 50-175 um and any range or value therein) can be maximized using the nanoparticles described herein. In one embodiment, the invention comprises (i) nanoparticles sized in the range of about 10 to about 300 nm (e.g., 10-250, 20-100, 50-250, 100-200, 100-150 mm and any range or value therein) may be generated, (ii) a cosmetically acceptable carrier and/or a pharmaceutically acceptable carrier, and (iii) a drug or agent including, but not limited to benzoyl peroxide, salicylic acid, other drugs, biologic compounds or agents, and combinations of two, three or more.

### Silver Nanoplate Synthesis

In one embodiment, silver nanoplates will be synthesized using silver seeds prepared through the reduction of silver nitrate with sodium borohydride in the presence of sodium citrate tribasic and poly sodium styrene sulfonate under aqueous conditions. Silver seed preparation: 21.3 mL of an aqueous 2.5 mM sodium citrate tribasic solution will be allowed to mix under magnetic stiffing. 1 mL of a 2 g/L poly styrene sodium sulfonate (PSSS) solution will be prepared in a separate beaker. 21.3 mL of a 0.5 mM silver nitrate solution will then prepared by dissolving the salt in water. Once the above solutions are prepared, 1.33 mL of a 0.5 mM sodium borohydride solution will be prepared in 4° C. water. The borohydride and PSSS solutions will then be added to the beaker containing the citrate and allowed to mix. The silver nitrate solution will then pumped into the citrate solution using a peristaltic pump at a rate of 100 mL/min. This seed solution will then be allowed to stir overnight at room temperature. Silver nanoplates will be prepared by mixing 1530 mL Milli-Q water with 35 mL of a 10 mM ascorbic acid solution. Once the solution is sufficiently mixed, the prepared silver seed will be added to the reactor. 353 mL of a 2 mM silver nitrate solution will be pumped into the reactor at a rate of 100 mL/min (or rates +/−10%, 20%, 25%, 50%, 100% or more). The reaction will be mixed for two hours (e.g., +/−10, 15, 20, 30, 45, 60 minutes). TEM analysis should show that over 70% of the particles are nanoplates. In other embodiments, over 50%, 60%, 80%, 90% of the particles will be nanoparticles. The optical density of the solution will be 2.8 cm⁻¹. In various embodiments, the optical density will be in the range of 0.05 to 50 cm⁻¹ (e.g., 0.1-10, 1-5, 2-4 cm⁻¹ or any value or range therein).

### Silver Nanoplate Concentration

In one embodiment of concentrating silver nanoplates, 1.2 L of silver nanoplates with a peak optical density of about 4 cm⁻¹ will be mixed with 4 L of anhydrous ethanol and about 49 mL of ammonium hydroxide solution. 0.6 mL of a dilute aminopropyltriethoxysilane (APTES) will be added to the solution. After 15 minutes of incubation, 6.5 mL of tetraethylorthosilicate (TEOS) solution will be added. After 24 hours 1 L of the solution will be concentrated using a 500 kD polysulfone tangential flow membrane with 1050 cm of surface area. The final solution volume will be decreased to 150 mL (or 100-500 mL, 100-200 mL, and any value or range therein), increasing the silver nanoparticle solution optical density to about 40 cm⁻¹. In various embodiments, the optical density will be in the range of 1-200 cm⁻¹ (e.g., 1-100, 25-150, 50-125 cm⁻¹ or any value or range therein).

Thus, according to one embodiment, a method for increasing a silver nanoplate solution from 4 cm⁻¹ to 40 cm⁻¹ (e.g., an increase of roughly 10 times the optical density, or in various embodiments, an increase of 1-100×, 2-50×, 5-20×, or any range or value therein) will comprise the steps of adding anhydrous ethanol, ammonium hydroxide solution, aminopropyltriethoxysilane (APTES), and/or tetraethylorthosilicate (TEOS) to the silver nanoplates, and concentrating the solution with tangential flow filtration.

Nanoplates with a Silica Shell

In one embodiment, a silica shell will be grown on the surface of 800 nm resonant (˜75 nm edge length) polyvinylpyrrolidone (PVP) capped silver nanoplates. 400 mL of a solution of 800 nm resonant PVP capped silver nanoplates at a concentration of 2 mg/mL (20 cm⁻¹ O.D.) will be added to 2.3 L of reagent grade ethanol and 190 mL Milli-Q water under constant stirring. 4.3 mL of dilute aminopropyl triethoxysilane (215 uL APTES in 4.085 mL isopropanol) will then be added to the solution, followed (e.g., immediately) by the addition of 44 mL of 30% ammonium hydroxide. After 15 minutes of incubation, 31 mL of dilute tetraethylorthosilicate (1.55 mL TEOS in 29.45 mL isopropanol) will be added to the solution. The solution will then be left to stir overnight (or in various embodiments, 1-18, 3-12, 5-10, 6-8 hours, or any value or range therein). The nanoplates will then be centrifuged on an Ultra centrifuge at 17000 RCF for 15 minutes and reconstituted in Milli-Q water each time and repeated twice. The silica shell thickness will be 15 nm (or in various embodiments, 1-50, 5-25, 10-20 nm, or any value or range therein). The optical density of the concentrated material will be 2040 cm⁻¹. In various embodiments, the optical density of the concentrated material will be 100-5000 cm⁻¹. (e.g., 750-4000, 1000-3000, 1500-2500 cm⁻¹ and any value or range therein).

Composition with One or More Agents (Such as Drugs)

Concentrated nanoplates will be combined with a cosmetically and/or pharmaceutically acceptable carrier and a drug (or other agent) thereby forming a nanoparticle drug composition. In some embodiments, the drug will include one or more of: glycolic acid, sulfur, salicylic acid, and/or benzoyl peroxide. Other drugs from the acne monograph may also be used. In various embodiments, a drug carrier will be synthesized from a water base with varying amounts of a material (e.g. salicylic acid, benzoyl peroxide, glycolic acid, sulfur, vitamin A, vitamin B, vitamin C, vitamin D, vitamin K, etc.) in an effective amount (e.g., 1%, 2%, 3%, 4%, 5%, 6%, 7%, 8,%, 9%, 10%, 15%, 20%), propylene glycol (e.g., 10%, 20%, 22%, 25%, 30%), surfactant (e.g. sodium dodecyl sulfate) in an amount (1%, 1.1%, 1.2%, 1.5%. 2%. 5%), preservative (e.g. PE9010) in an amount (e.g., 1%, 1.1%, 1.2%, 1.5%. 2%. 5%), and a thickener (e.g. carbopol) to achieve a desired viscosity (e.g., 3000 cP, 3500 cP, 4000 cP). This drug carrier will be mixed with a concentrated plasmonic nanoplate solution (e.g., optical density of 10, 20, 50, 75, 100, 125, 150, 175, 200, 250, 300, 400, 500 O.D.) in water in various ratios (e.g., 1:1 ratio 1 part particles, 1 part drug carrier, 1:2; 1:3, 1:4; 1:5; 1:10; 10:1, 5:1; 4:1, 3:1; 2:1, etc.) to form a nanoparticle drug composition. In various embodiments, heating of a drug with the nanoparticles will increase the temperature of the drug and/or targeted tissue in the vicinity and/or in contact with the nanoparticles by 1, 2, 3, 4, 5, 7, 10, 15, 20, 25, 30, 35, 40, 45, 50, 55, 60, 65, 70, 75, 80 degrees Celsius, or more, and any temperature ranges therein.

In one embodiment, a drug carrier will be synthesized from a water base with 22% propylene glycol, 6% Salicylic acid, 1.1% surfactant (e.g. sodium dodecyl sulfate), 1.1% preservative (e.g. PE9010), and a thickener (e.g. carbopol) to achieve a desired viscosity (e.g., 3500 cP). This drug carrier will be mixed with a concentrated nanoplate solution in water in a 1:1 ratio (1 part particles, 1 part drug carrier) to form a nanoparticle composition for acne treatment.

### Example 2: Use of Plasmonic Nanoparticles for Ablation of Acne

In one embodiment, a composition described herein will be used as a topical in the treatment of acne. In one embodiment, about 1 to 10 ml of the formulation will be applied to an acne site (e.g., on the face, neck, body) once a week until acne symptoms subside (lesion counts reduce). The formulation will be targeted into to the sebaceous follicles (pilosebaceous units) by massaging the solution by hand or with a mechanical vibration device (e.g., a device vibrating at about 80 Hz). Excess solution on the surface of the facial skin will be removed with a cleansing wipe. The remaining solution in the sebaceous follicles will then be activated with light energy. In one embodiment, the light energy will be in the violet, red, or infrared and will be provided by an Intense Pulsed light (IPL) device operating at 1-20 J/cm² and 1-5 ms pulse width. The IPL device will be passed along the skin to illuminate all target areas of the face to be treated. In one embodiment, a diode operating in the violet, red, or infrared will be used to activate the solution. Continuous or pulsed light may be used to target heating to the pilosebaceous unit and increase the permeability of the tissue and activity of salicylic acid (or other agent) in the tissue. Clearance of acne lesions and improved skin health will be achieved within 3-4 weeks of treatment (or in various embodiments, 1-10 weeks, 2-5 weeks, or any value or ranges therein). In one embodiment, the composition will be applied to any skin (e.g., face, neck, head, body, chest, back, etc.) with acne.

### Example 3: Formulation of Plasmonic Nanoparticles to Treat an Infection

In one embodiment, a composition described herein will be used as a topical formulation in the treatment of an infection. In one embodiment, about 1-5 ml of the formulation will be applied to the skin proximate an infection once a week until the infection symptoms subside. In one embodiment a therapeutically effective amount of a material will be applied to an infection.

In one embodiment, about 1 ml of the formulation will be applied to the infection site once a week until infection symptoms subside. The formulation will be targeted into to the infection site and tissue proximate the infection site by massaging the solution by hand or with a mechanical vibration device (e.g., a device vibrating at about 80 Hz). Excess solution on the surface of the infection site (and tissue around the infection site) will be removed with a cleansing wipe. The remaining solution at the infection site will then be activated with light energy. In one embodiment, the light energy will be in the violet, red, or infrared and will be provided by an Intense Pulsed light (IPL) device operating at 1-20 J/cm² and 1-5 ms pulse width. The IPL device will be passed along the skin to illuminate all target areas of the infection site to be treated. In one embodiment, a diode operating in the violet, red, or infrared will be used to activate the solution. Continuous or pulsed light may be used to target heating to the infection site the permeability of the tissue and activity of a drug (or other agent) in the tissue. Clearance of the infection site and improved skin health will be achieved within 3-4 weeks of treatment (or in various embodiments, 1-10 weeks, 2-5 weeks, or any value or ranges therein). In one embodiment, the composition (e.g., a drug formulation) will be applied to any skin (e.g., face, neck, head, body, chest, back, etc.) with an infection. In one embodiment, the composition will be applied to any tissue (e.g., face, neck, head, body, chest, back, etc.) with an infection.

In one embodiment, plasmonic nanoparticles will be applied to an infection. In one embodiment, an agent will be encapsulated with a nanoparticle and applied to an infection site. In some embodiments, the nanoparticles will be coated with a hydrophilic coating. Energy, e.g., light at a wavelength of 440 nm, 640 nm, 755 nm, 810 nm, or 1064 nm, will be applied to activate a plasmon in the plasmonic nanoparticles, thereby activating the material applied to the infection, thereby treating the infection. In some embodiments, the nanoparticles will be coated with a hydrophilic coating. In some embodiments, the nanoparticles and agent will be in a composition with a cosmetically and/or pharmacologically acceptable carrier. In one embodiment, the nanoparticle will comprise gold. In one embodiment, the nanoparticle will comprise silver. In one embodiment, the nanoparticles will have a concentration of 10⁹. In one embodiment, the nanoparticles will have a concentration of 10¹⁰. In one embodiment, the nanoparticles will have a concentration of 10¹¹. In one embodiment, the nanoparticles will have a concentration of 10¹². In one embodiment, the nanoparticles will have a concentration of 10¹³. In one embodiment, the nanoparticles will have a concentration of 10¹⁴. In one embodiment, plasmonic nanoparticles and a therapeutic material will be dissolved in a solution and applied to an infection site to treat the infection site with the application of light and/or heat energy. In some embodiments, the light will have a visible spectrum wavelength. In some embodiments, the light will have an infrared wavelength.

### Example 4: Delivery of a Formulation of Plasmonic Nanoparticles for Treatment

In one embodiment, a composition described herein will be used as a topical formulation for a cosmetic or a therapeutic treatment. In some embodiments, the composition will comprise organic material. In some embodiments, the composition will comprise inorganic material. In some embodiments, the composition will comprise a solid. In some embodiments, the composition will comprise a liquid. In some embodiments, the composition will comprise a gas. In some embodiments, the composition will comprise bubbles. In some embodiments, the composition will be subjected to a phase change. In some embodiments, the composition will be stirred. In some embodiments, the composition will be centrifuged. In some embodiments, the composition will be filtered. In some embodiments, the composition will be thickened. In some embodiments, the composition will be thinned. In some embodiments, the composition will have increased viscosity. In some embodiments, the composition will have decreased viscosity. In some embodiments, the composition will be refined. In some embodiments, the composition will be stabilized. In one embodiment, mechanical vibration will be used to assist in the targeted delivery of the composition to an infection site for treatment. In one embodiment, acoustic vibration will be used to assist in the targeted delivery of the composition to an infection site for treatment. In one embodiment, ultrasound will be used to assist in the targeted delivery of the composition to an infection site for treatment. In one embodiment, suction will be used to assist in the targeted delivery of the composition to an infection site for treatment. In one embodiment, air pressure will be used to assist in the targeted delivery of the composition to an infection site for treatment.

In one embodiment, the composition will be placed inside the body (e.g., injected, inserted via catheter, swallowed in a pill, etc.). Treatment will be provided with an energy source that is applied inside the body, e.g., via an ingested energy source, an energy source delivered via catheter, or other delivery system.

### Example 5: Transdermal Drug Patch Activation with Plasmonic Nanoparticles

In one embodiment, a transdermal patch with a drug (e.g., therapeutic material or other agent) and plasmonic nanoparticles will be applied to a tissue surface (e.g., a skin surface). In one embodiment, a wavelength of light energy will be applied to the transdermal patch, thereby activating the drug and enhancing the delivery of the drug to the tissue. In one embodiment, a transdermal patch will provide for controlled release of the drug into the tissue surface at a controllable rate, as activated by energy delivered to the plasmonic nanoparticles. In one embodiment, a portion of the transdermal patch will be transparent to light at one or more wavelength ranges. In one embodiment, the transparent portion of the transdermal patch will be configured to correspond to a treatment area, e.g., a scar, an incision, a wound, a tattoo, etc. In one embodiment, a photograph will be taken of a target skin treatment site. The image of the target skin treatment site will be printed to create a stencil with a transmission region (e.g, transparent or transmission portion) configured to transmit 100%, 99%, 98%, 97%, 95%, 90%, 85%, 80%, 75%, 70%. 60%, 50% or less (and any range between 1-100%) through the transdermal patch. In one embodiment, the image will be an inverse of the target skin treatment site. In one embodiment, the image will be attached to the transdermal patch. The transdermal patch will be configured to provide a selective, controlled level of drugs to the tissue at the tissue surface and in some embodiment, below the tissue surface via pores, hair follicles, and/or increased skin permeability. The transdermal patch will be left on the tissue surface provide multi-hour and/or multi-day dosing that is convenient for the patient. In one embodiment, the rate of drug delivery will be controlled by the activation of the plasmonic nanoparticles. In one embodiment, the amount of adhesion in a transdermal patch will be controlled by the activation of the plasmonic nanoparticles.

### Example 6: Monitoring of a Formulation of Plasmonic Nanoparticles for Treatment

In one embodiment, a composition described herein will be used as a topical formulation for treatment, and will be monitored with a monitoring device. In one embodiment, a treatment site will be monitored for treatment. In one embodiment, an animal skin sample (3 cm×3 cm) will be treated, the animal will be sacrificed, and the skin sample will be excised and examined under a microscope to measure improvement of the skin sample in view of the treatment.

In some embodiments, the composition comprises one or more of the following: means for generating localized heat (e.g., photoactive particles such as nanoparticles and other particles as described herein); means for delivering energy (e.g., light, laser and other energy sources as described herein); and means for providing a therapeutic effect (e.g., one or more agents as described herein).

In some embodiments, the composition comprises various features that are present as single features (as opposed to multiple features). For example, in one embodiment, the composition includes a single type of plasmonic nanoparticle with a single agent (e.g., drug). The composition may be mixed to include the nanoparticles and the agent. A single surfactant or a single cosmetically or therapeutically acceptable carrier may also be included. Multiple features or components are provided in alternate embodiments.

### Example 7: Enhanced Delivery of a Skin Lightening, Epilation or Skin Tightening Agent

In one embodiment, a composition described herein will be used as a topical treatment with a skin lightening agent, and epilation (hair removal) agent or a skin tightening agent. In one example, a composition containing nanoparticles at a concentration of 10¹⁰ to 10¹⁴ will be provided with a skin lightening agent, an epilation agent or a skin tightening agent at a therapeutically effective amount (e.g., 0.05-25% per ml of the composition, or % m/m, % m/v, or % v/v of the composition).

The skin lightening agent, as an example, comprises an anti-melanin that reduces the production or storage of melanin, increases melanin degradation, and/or decreases the melanin transport from melanocytes to keratinocyte.

With respect to the epilation agent, through the combination of the photoactive particles and the agents described herein, removal of light or unpigmented hairs and/or removal of darker hair on darker skin are provided in multiple embodiments.

The skin tightening agent, as an example, comprises one or more compounds that aid in generating or repairing collagen and/or elastin. Vitamins and minerals are used as an example (such as copper, vitamin C, zinc, niacinamide, vitamin A, and combinations thereof). The skin tightening agent, as an example, can also comprises a plumper or other molecule that enhances fullness such as hyaluronic acid and/or sodium hyaluronate.

The nanoparticles and the agent will be provided either in one container or in two separate containers. The nanoparticles will include nanoplates or nanospheres that have a metal portion (either gold or silver) having at least one dimension in the range of 100-200 nm and a concentration of 10¹⁰ to 10¹⁴ per ml of the composition. The metal portion can form the core or a non-core layer. The nanoparticles will be coated with silica, PEG, or other suitable coating that facilitates selective removal from the skin. In one embodiment, about 1-15 ml of the formulation will be applied to the skin proximate a treatment site once a week until the symptoms relating to the treatment subside. The formulation will be targeted into to the treatment site and tissue proximate the treatment site by massaging the solution by hand or with a mechanical vibration device (e.g., a device vibrating at about 80 Hz). Excess solution on the surface of the treatment site (and tissue around the treatment site) will be removed with a cleansing wipe. The remaining solution at the treatment site will then be activated with light energy. In one embodiment, the light energy will be in the violet, red, or infrared and will be provided by an Intense Pulsed light (IPL) device operating at 1-20 J/cm² and 1-5 ms pulse width. The IPL device will be passed along the skin to illuminate all target areas of the infection site to be treated. In one embodiment, a diode operating in the violet, red or infrared will be used to activate the solution. Continuous or pulsed light may be used to target heating to the infection site the permeability of the tissue and activity of a drug (or other agent) in the tissue. Resolution of the treatment site and improved skin health will be achieved within 3-4 weeks of treatment (or in various embodiments, 1-10 weeks, 2-5 weeks, or any value or ranges therein). In one embodiment, the agent composition will be applied to any skin or tissue (e.g., face, neck, head, body, chest, back, etc.) with the treatment site. Improvement may also be seen the same day of treatment.

In one embodiment, plasmonic nanoparticles will be applied to a treatment site. In one embodiment, an agent will be encapsulated with a nanoparticle and applied to an infection site. In some embodiments, the nanoparticles will be coated with a hydrophilic coating. Energy, e.g., light at a wavelength of 440 nm, 640 nm, 750 nm, 8100 nm, 1064 nm, will be applied to activate a plasmon in the plasmonic nanoparticles, thereby activating the material applied to the treatment site, thereby treating the treatment site. In some embodiments, the nanoparticles will be coated with a hydrophobic coating. In some embodiments, the nanoparticles and agent will be in a composition with a cosmetically and/or pharmacologically acceptable carrier. In one embodiment, the nanoparticle will comprise gold. In one embodiment, the nanoparticle will comprise silver. In one embodiment, the nanoparticles will have a concentration of 10⁹, 10¹⁰, 10¹¹, 10¹², 10¹³, or 10¹⁴, or any range therein. In one embodiment, plasmonic nanoparticles and a therapeutic material will be mixed in a solution and applied to a treatment site to treat the treatment site with the application of light and/or heat energy. In some embodiments, the light will have an ultraviolet spectrum wavelength. In some embodiments, the light will have a visible spectrum wavelength. In some embodiments, the light will have an infrared wavelength.

It is intended that the specification (including the claims, examples and drawings) be considered as disclosing non-limiting embodiments of the invention. It should be understood that the invention(s), also includes modifications, equivalents, and alternatives falling within the spirit and scope of the various embodiments described herein and the appended claims. Any methods disclosed herein need not be performed in the order recited. The methods disclosed herein include certain actions taken by a practitioner; however, they can also include any third-party instruction of those actions, either expressly or by implication. For example, actions such as “identifying a target region of skin tissue” include “instructing the identification of a target region of skin tissue.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” or “substantially” include the recited numbers. For example, “about 3 mm” includes “3 mm” The terms “approximately”, “about”, and “substantially” as used herein represent an amount or characteristic close to the stated amount or characteristic that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount or characteristic.

1.-69. (canceled)

70. A method of using nanoparticles for increasing kinetics of a drug reaction with a transdermal patch, comprising:
applying a transdermal patch comprising a composition to a skin surface,
wherein said composition comprises a drug and a plurality of plasmonic nanoparticles,
wherein the plasmonic nanoparticles comprise a metal portion and a coating;
wherein the transdermal patch comprises a portion configured for transmission of light through the transdermal patch; and
irradiating the composition with a light source thereby inducing a plurality of surface plasmons in said plasmonic nanoparticles,
wherein inducing the plurality of surface plasmons generates localized heat in a target tissue proximate the skin surface, thereby increasing a temperature of the target tissue, thereby increasing kinetics of a drug reaction at the target tissue.

71. (canceled)

72. (canceled)

73. A method of claim 70, wherein the composition comprises any one or more of growth factors, collagen byproducts, collagen precursors, hyaluronic acid, glucosamine, allantoin, vitamins, oxidants, antioxidants, amino acids, retinoids, retinoid-like compounds, and minerals.

74. A method of claim 70, wherein the composition comprises any one or more of steroidal anti-inflammatory drugs, non-steroidal anti-inflammatory drugs, antioxidants, antibiotics, antiviral drugs, antiyeast drugs and antifungal drugs.

75. A method of claim 70, wherein the drug comprises any one or more of glycolic acid, sulfur, salicylic acid, and benzoyl peroxide.

76. A method of claim 70, wherein the temperature of the target tissue effects kinetic reactions based on the Arrhenius equation.

77. A method of claim 70, wherein the temperature of the target tissue is increased by 1-30 degrees Celsius.

78. (canceled)

79. (canceled)

80. (canceled)

81. A method of claim 70, wherein the distributing the composition comprises activation of a mechanical vibration, further comprising pre-treating the skin surface prior to applying of the transdermal patch on the skin surface, wherein pre-treating the skin surface comprises hair removal.

82. A method of claim 70, wherein the distributing the composition comprises activation of a mechanical vibration, wherein the plasmonic nanoparticles comprise an optical density of 10 O.D. to 500 O.D. at a light range from 700 nm to 1200 nm.

83. A method of claim 70, wherein the distributing the composition comprises activation of a mechanical vibration, wherein the plasmonic nanoparticles comprise a solid, conducting silver core and a silica coating.

84. (canceled)

85. A method of claim 70, wherein the drug is provided in a therapeutically or cosmetically effective amount.

86. A method of claim 70, wherein the drug comprises salicylic acid.

87. A method of claim 70, wherein the drug comprises benzoyl peroxide.

88. A method of claim 70, wherein the drug comprises glycolic acid.

89. A method of claim 70, wherein the drug comprises sulfur.

90. A method of claim 70, wherein the drug comprises 5-ALA.

91. A transdermal patch, comprising:
a transmission portion configured for transmission of light through the transdermal patch;
an adhesive configured for application of the transdermal patch to a skin surface;
a composition comprising a drug and a plurality of plasmonic nanoparticles,
wherein the transmission portion is configured for transmission of sufficient light to induce a plurality of surface plasmons in said plasmonic nanoparticles to generate localized heat in a target tissue proximate the skin surface, thereby increasing a temperature of the target tissue, thereby increasing kinetics of a drug reaction at the target tissue.

92. The transdermal patch of claim 91, wherein the transmission portion is configured to transmit between 80% and 100% of light through the transdermal patch.

93. The transdermal patch of claim 91, wherein the transmission portion is configured to transmit between 90% and 100% of light through the transdermal patch.

94. The transdermal patch of claim 91, wherein the transmission portion comprises an image of a treatment region on the skin surface.

95. The transdermal patch of claim 91, wherein the transmission portion comprises an outline of a treatment region.

96.-111. (canceled)
